(19) United States
(12) Patent Application Publication
(10) Pub. No.: US 2008/0103387 A1
(43) Pub. Date: May 1, 2008

(54) Breast Cancer Detection And Biopsy

(75) Inventor: Jacob Gross, Even-Yehuda (IL)

Correspondence Address: Martin D.Moynihan;Prtsi Inc, P.O.BOX 16446, Arlington, VA, 22215, US

(73) Assignee: Scientific Biospy Ltd., Even-Yehuda (IL)

(21) Appl. No.: 11/662,883

(22) PCT Filed: Sep. 15, 2004
(86) PCT No.: PCT/IL04/00854

§ 371 Date: Mar. 15, 2007

(51) Int. Cl. A61B 6/04; A61B 10/02

(52) U.S. Cl. 600/424; 378/37; 600/564

(57) ABSTRACT

A breast compression device suitable for compressing a breast for an x-ray guided biopsy, the device comprising:
a) a base for contacting a portion of the surface of the breast;
b) a flexible band, anchored to the base, adapted to be positioned on a side of the breast opposite the base, when the breast is inserted between the base and the band, which band is adapted to be wrapped at least part way around the breast; and
c) a tensioning device, adapted to tension the flexible band around the inserted breast when the flexible band is anchored to the base, thereby compressing the breast between the flexible band and the base.

A system for performing a biopsy of a breast lesion of a patient in a reclining position, the system comprising:
a) a chair with a back that has an upright position and at least one reclining position;
b) a compression device adapted to hold a breast of the patient seated in the chair in a compressed state, when the chair back is in the upright position or in at least one reclining position; and
c) a biopsy unit having a biopsy element and being coupled to the chair back at least in a reclining position.

### FIELD OF THE INVENTION

The field of the invention is devices and methods for breast biopsy.

### BACKGROUND OF THE INVENTION

Breast biopsy may be performed either in open surgery or with needle biopsy. Needle biopsy may be guided either using ultrasound, with free hand positioning, or, in the case of micro-calcifications and other lesions which cannot be seen with ultrasound, using stereotaxic x-ray systems, sometimes called “stereotactic” x-ray systems.

Two types of stereotaxic systems for breast biopsy currently exist: add-on biopsy systems used in conjunction with mammography machines, in which the patient sits upright, and prone tables dedicated only to biopsy in which the patient lies face down. In upright biopsy systems the large core needle system may be seen by the patient. These systems are not suitable for patients who have a tendency to faint, or to be frightened at the sight of long needles being inserted into their bodies.

A prone table biopsy system is described, for example, by Jan Bolmgren, Bertil Jacobson and Bjorn Nordenstrom, “Stereotaxic Instrument for Needle Biopsy of the Mamma,” Am. J. Roentgenol. 129:121-125 (July 1977), incorporated herein by reference. The patient lies in a face down prone position on the table, while the breast on which the biopsy is to be performed hangs down through a hole in the table, and is immobilized by clamping it between a compression plate, made of polycarbonate, and a breast support plate, typically made of carbon fiber reinforced plastic. Two x-ray images, taken from different angles, typically 15 degrees on opposite sides of the normal to the film plane, are used to calculate the three-dimensional coordinates of the lesion with respect to the compression plate. With the breast still clamped in the same position, the biopsy is performed through a small biopsy window, typically 50 mm by 50 mm, in the compression plate.

The biopsy system described in the Bolmgren et al paper has several disadvantages in regard to the comfort of the patient. The prone position can cause neck spasm or back pains, and is problematic for women with large bellies. There may be no comfortable place for the patient to put her hands. The patient is largely constrained from moving during the procedure. Because the breast is now hanging down, the lesion may be in a different relative location than when the original diagnostic mammogram was made in an upright position. Hence it may take a long time to properly position the breast so that the lesion falls within the biopsy window, and to properly position the needle within the window, further increasing the length of the procedure and patient discomfort.

Because the force of compression is usually uncontrolled and unknown, there may be insufficient compression, resulting in movement of the breast, or too much compression, which causes unnecessary discomfort. In any case, it is difficult to immobilize the breast completely, due to the flat shape of the compression plate and small size of the window, and the fact that the patient can still move up slightly. Another disadvantage of a prone table biopsy system is that, depending on the angle of orientation of the biopsy needle, it may be impossible to perform biopsies on lesions that are too close to the chest wall, due to the thickness of the table. Furthermore, such systems usually cannot be used with ultrasound guidance, because ultrasound does not couple well between the rigid compression plate and the breast tissue. The patient tends to lose a significant amount of blood during the biopsy when the breast is hanging down. Finally, the prone position has the possible psychological disadvantage that the patient is not facing the doctor who is performing the procedure.

U.S. Pat. No. 5,386,447, to Siczek, describes a mammography and biopsy apparatus which is initially vertical, and with the patient standing on it. The patient holds on to a bar above her head, and is further held in place by a strap around her waist. The apparatus has conventional compression plates. Without releasing the breast from the compression plates, the whole apparatus may be tilted forward by 90 degrees, so that the patient is in the prone position. It additionally may even be tilted forward slightly past the prone position, or slightly back past the vertical position.

U.S. Pat. No. 6,557,196, to Falbo, Sr. et al, describes a mammography and biopsy apparatus in which the patient lies on her side.

Several patents describe devices which compress the breast for mammography, but also allow ultrasound to couple to the breast for ultrasound imaging and guidance. For example, U.S. Pat. No. 6,547,499, to Dines et al, describes an upper compression plate which is slightly flexible, and can conform slightly to the shape of the breast. Alternatively, an elastic membrane is pushed against the breast from above. The compression plate or membrane contacts the breast over an extended area, and may be used to transmit ultrasound. U.S. Pat. No. 6,682,484, to Entrekin et al, describes a breast compression system, also suitable for both x-ray and ultrasound imaging, in which the lower compression plate is replaced by a flexible membrane under tension, stretched so that it is nearly flat. German patent publication DE 19901724 describes a mammography apparatus in which fluid filled bags surround and conform to the breast, transmitting the force of flat compression plates as well as ultrasound waves to the breast.

Fluid-filled bags or reservoirs surrounding the breast for ultrasound imaging are also described in U.S. Pat. No. 6,128,523 and German patent publication DE 19610802, while gas-filled bags surrounding the breast for optical imaging are described in U.S. Pat. No. 6,587,578, but in these cases the breast is not compressed sufficiently for x-ray imaging.

A number of patents describe rigid compression plates for mammography or breast biopsy which are curved to conform to the shape of the breast (U.S. Pat. No. 4,943,986 to Barbarisi), or are flat but are not parallel (U.S. Pat. No. 6,577,703 to Lindstrom et al, and WO 03/041586 to Demay et al), or are lined with soft padding (U.S. Pat. No. 6,577,702 to Lebovic et al), to provide greater comfort or more uniform compression. There are also patents, for example U.S. Pat. No. 6,304,770, describing soft or conforming breast stabilization devices that do not compress the breast, and are used, for example, for ultrasound-guided biopsies.

U.S. Pat. No. 6,418,188, to Broadnax, describes an elastomeric cup which covers the whole breast, and compresses it for mammography. An eyelet, attached to the end of the cup which covers the nipple region, is used to hold the breast up if the patient is standing. Alternatively, the patient is in the prone position and the breast hangs down, in which case the eyelet is not needed.

The above mentioned patents and publications are all incorporated herein by reference.

### SUMMARY OF THE INVENTION

An aspect of an embodiment of the invention concerns a soft breast compression device used to immobilize the breast when performing a biopsy. The soft compression device comprises a thin, flexible sheet of a material, for example nylon, which goes at least part of the way around the breast, conforming to the curved surface of the breast, and is then pulled tightly around the breast with a tensioning mechanism. Although the degree of compression of the breast provided by the soft compression device may or may not be sufficient to provide optimal diagnostic x-rays of the breast, the degree of compression is sufficient to provide x-ray images for guiding a biopsy of a known lesion. Optionally, the tensioning mechanism is attached to a relatively rigid support which goes under the breast, and optionally the tensioning mechanism allows the angle between the flexible sheet and the rigid support to be adjusted to the shape of the breast. Optionally, the soft compression device does not cover the nipple.

The soft compression device applies pressure around most of the breast, in contrast to conventional compression devices using flat compression plates, in which the pressure is concentrated in a relatively small part of the breast, so the soft compression device is more comfortable for the patient, and provides better immobilization. Furthermore, ultrasound can be transmitted through the soft compression device, instead of or in addition to x-rays, for providing images for locating the lesion. Optionally, the biopsy needle goes through the soft material of the compression device, so the biopsy needle can come in from any direction, rather than being limited to a small window, and can reach lesions even if they are close to the chest wall. Optionally, if used for a biopsy, the soft compression device is not an integral part of the unit used to perform the biopsy, but is separable from the biopsy unit, and is first placed around the breast and tightened, and then attached to the biopsy unit.

An aspect of an embodiment of the invention concerns a stereotaxic x-ray biopsy system in which the patient is seated in a chair that leans back. A compression device, for example the soft compression device described above or another breast compression device known in the art, compresses the breast, optionally while the patient is sitting upright. The breast remains compressed in the compression device while the patient leans back in the chair to a comfortable angle. Alternatively, particularly for patients with large breasts, the breast is not compressed until after the patient leans back, at least a little.

Once the patient is leaning back, or even before that, the compression device with the captured breast is optionally coupled in a spatially stable way to a unit with a biopsy needle which performs the biopsy, guided by stereotaxic x-ray images made of the breast in the compression device, made by an x-ray unit. Alternatively, the compression device is already coupled to the biopsy unit when the breast is compressed, or is permanently coupled to the biopsy unit.

Optionally, the biopsy unit and/or the x-ray unit are attached to the chair, so that when the chair leans back at any of a range of angles, the biopsy unit is in a proper position relative to the breast to perform the biopsy, and the x-ray unit is in a proper position to produce stereotaxic x-ray images of the breast. Alternatively, the biopsy unit and x-ray unit are attached to a separate stand located adjacent to the chair, and the stand holding the biopsy unit and x-ray unit tilts back to the same angle as the back of the chair, when the chair leans back, keeping the biopsy unit and x-ray unit approximately in the same position relative to the back of the chair, when the chair leans back. Adjustments are optionally made to compensate for small changes in the position of the biopsy unit and x-ray unit relative to the back of the chair. Even if the biopsy unit is attached to the chair, adjustments in the relative position of the biopsy unit are optionally made according to the position of the patient's breast relative to the chair, which may change when the patient leans back, even after the breast is compressed. The patient is generally free to move any part of her body other than the breast being biopsied.

This system, particularly when used with the soft compression device, avoids most of the sources of patient discomfort in a conventional prone table biopsy system. This system also avoids some problems of conventional upright biopsy systems. It is suitable for patients who have a tendency to faint, since the patient is reclining. If the patient tilts her head back slightly, which is not uncomfortable to do when reclining, then a curtain can be put between the patient's face and the biopsy unit, for patients who have a tendency to faint at the sight of a long needle.

Optionally, the stereotaxic x-ray images are recorded using a digital x-ray detector which is located at a known stable position relative to the biopsy unit and the compression device, and the digital images are optionally transferred automatically to a controller of the position of the biopsy needle. Once the location of the lesion is determined on each stereotaxic image, for example by having a physician indicate where the lesion is located on a display screen, the controller optionally uses the digital images to calculate the precise three-dimensional location of the lesion, and automatically directs the biopsy needle to the lesion. Optionally, another pair of digital stereotaxic x-ray images, or a single x-ray image, is then made to verify that the biopsy needle is indeed located at the correction position relative to the lesion, and optionally any needed corrections are made automatically by the controller, using the new digital images. This procedure has the potential advantage, compared to conventional biopsy systems using x-ray film, that the patient may not have to wait such a long time from the time her breast is compressed until the biopsy in completed.

An aspect of an embodiment of the invention concerns a device and method for making x-ray images of the breast in which the patient is seated in a chair that leans back, even without doing a biopsy. The x-ray images are used for detecting and/or diagnosing lesions in the breast, for example. The x-rays are generated by an x-ray unit which is optionally attached to the chair, so that the x-ray unit remains in the same position and orientation relative to the breast when the chair leans back. A compression device immobilizes the breast while the patient is sitting upright, and the breast remains immobilized when the chair leans back. An x-ray detector is optionally attached to the chair, or to the compression device, or both.

There is thus provided, in accordance with an exemplary embodiment of the invention, a breast compression device suitable for compressing a breast for an x-ray guided biopsy, the device comprising:
a) a base for contacting a portion of the surface of the breast;
b) a flexible band, anchored to the base, adapted to be positioned on a side of the breast opposite the base, when the breast is inserted between the base and the band, which band is adapted to be wrapped at least part way around the breast; and
c) a tensioning device, adapted to tension the flexible band around the inserted breast when the flexible band is anchored to the base, thereby compressing the breast between the flexible band and the base.

Optionally, the flexible band is adapted to be wrapped at least half way around the breast.

Optionally, the base is relatively rigid.

Optionally, the base is flat where it contacts the breast.

Alternatively, the base is curved to conform to the breast.

Optionally, the device is adapted to compress the breast with a force between 1 and 5 kilograms.

Alternatively, the force is between 5 and 12 kilograms.

Alternatively, the force is greater than 12 kilograms.

In an embodiment of the invention, the device does not cover a region around the nipple when compressing the breast.

Optionally, the flexible band comprises nylon.

Optionally, the flexible band is elastomeric.

Optionally, the flexible band is capable of being punctured by a biopsy needle.

Optionally, the flexible band is adapted to having an area of the band removed, thereby exposing skin in that area and enabling a biopsy to be performed by puncturing the skin in that area.

Optionally, the device is configured so that the flexible band wraps a distance around the breast that is different for different positions along the axis of the breast, thereby allowing the compression device to better fit the contour of the breast and to compress it more uniformly.

Optionally, the device includes an anchor which attaches one or both ends of the flexible band to the base, which anchor is capable of rotating, relative to the base, around an axis extending to the left and right of the breast.

There is further provided, according to an exemplary embodiment of the invention, a system for performing a breast biopsy, the system comprising:
a) a breast compression device as described;
b) an ultrasound imaging unit adapted to obtain ultrasound images of the breast when the breast is compressed by the compression device; and
c) a biopsy element capable of being directed to a lesion in the breast, guided at least in part by the ultrasound images.

Optionally, the ultrasound imaging unit is adapted to obtain the ultrasound images by one or both of transmitting and receiving ultrasound waves through the flexible band.

There is further provided, according to an exemplary embodiment of the invention, a system for x-raying the breast, the system comprising:
a) a breast compression device as described; and
b) an x-ray unit and an x-ray detector adapted for producing x-ray images of the breast when the breast is compressed by the compression device.

Optionally, the system includes a biopsy unit adapted to be coupled to the compression device in a spatially stable way, with a biopsy element capable of being directed to a lesion in the breast, guided by the x-ray images.

Optionally, the system also includes a controller, wherein the x-ray detector and x-ray images are digital, the x-ray detector is adapted to send the x-ray images to the controller, and the controller is adapted to use the x-ray images to calculate the three-dimensional location of the lesion and to direct the biopsy element to said location.

Optionally, the system also includes an ultrasound imaging unit adapted to obtain ultrasound images of the breast when the breast is compressed by the compression device, wherein the biopsy element is capable of being guided in its initial approach to the lesion by the x-ray images and in its final approach to the lesion by the ultrasound images.

Optionally, the system also includes a chair with a back that has an upright position and at least one reclining position, wherein the compression device is adapted to hold the breast of a patient seated in the chair in a compressed state and in a stable position and orientation relative to the x-ray detector, at least in the reclining position.

There is further provided, according to an exemplary embodiment of the invention, a system for performing a biopsy of a breast lesion of a patient in a reclining position, the system comprising:
a) a chair with a back that has an upright position and at least one reclining position;
b) a compression device adapted to hold a breast of the patient seated in the chair in a compressed state, when the chair back is in the upright position or in at least one reclining position; and
c) a biopsy unit having a biopsy element and being coupled to the chair back at least in a reclining position.

Optionally, the biopsy unit is coupled to the chair back such that it remains in substantially the same position and orientation relative to the chair back when the chair back goes from the upright position to the at least one reclining positions, thereby enabling the biopsy to be performed in any of the at least one reclining positions.

There is further provided, in accordance with an exemplary embodiment of the invention, a system for performing a biopsy of a breast lesion of a patient in a reclining position, the system comprising:
a) a chair with a back that has an upright position and at least one reclining position;
b) a compression device adapted to hold a breast of the patient seated in the chair in a compressed state, when the chair back is in the upright position or in at least one reclining position; and
c) a biopsy unit having a biopsy element and configured to remain in substantially the same position and orientation relative to the breast of the patient when the chair back goes from the upright position to the at least one reclining positions, thereby enabling the biopsy to be performed in any of the at least one reclining positions.

Optionally, the system also includes an x-ray unit, configured to remain in substantially the same position and orientation relative to the breast of the patient when the chair back goes from the upright position to the at least one reclining positions, thereby enabling x-ray images to be made to guide the biopsy, in any of the at least one reclining positions.

Optionally, the chair comprises a base adapted to rest on a floor, and the x-ray unit and the biopsy unit are mounted on a stand adapted to rest on the floor in a stable position relative to the base of the chair.

In an embodiment of the invention, the biopsy unit is adapted to be coupled to the compression device in a manner allowing the biopsy unit to remain in a fixed position relative to the compression device.

Optionally, the biopsy unit is adapted to be rigidly coupled to the compression device.

Optionally, the coupling between the biopsy unit and the compression device is sufficiently stable to allow the biopsy element to be directed to the lesion when the breast is held in the compression device in the compressed state, guided by x-ray images of the breast made when the breast is held in the compression unit in the compressed state.

In an embodiment of the invention, the compression device comprises:
a) a soft compression device as described, adapted to hold a breast of the patient seated in the chair in a compressed state, when the chair back is in the upright position or in at least one reclining position; and
b) a hard compression device, adapted to further compress the breast when the breast is already held in the soft compression device and the chair back is in the reclining position, thereby enabling said x-ray images, if made when the breast is compressed by the hard compression device, to be of sufficient quality to guide the biopsy.

Optionally, the hard compression device comprises a flat, relatively rigid plate.

Optionally, the plate has a window, and the biopsy unit is adapted to perform the biopsy through the window.

Optionally, the system includes an x-ray detector coupled to one or both of the biopsy unit and the compression device, in a sufficiently stable way so as to enable the x-ray detector to be used for making x-ray images of the breast to guide the biopsy.

Optionally, the system includes a controller, wherein the x-ray detector and x-ray images are digital, the x-ray detector is adapted to automatically send the x-ray images to the controller, and the controller is adapted to use the x-ray images to calculate the three-dimensional location of the lesion and to direct the biopsy element to said location.

In an embodiment of the invention, the x-ray detector has a field of view that does not include the entire breast, and the x-ray detector is adapted to move to a plurality of positions, thereby enabling any of a plurality of zones of the breast to fall within its field of view, while the x-ray detector remains coupled to one or both of the biopsy unit and the compression device in the sufficiently stable way.

Optionally, the x-ray detector and the biopsy unit move together maintaining a same relative position, when the x-ray detector moves to a different one of the plurality of positions.

Optionally, the system includes an x-ray unit coupled to the chair at least in a reclining position.

In an embodiment of the invention, the biopsy unit is capable of being rotated into any of at least two different orientations, thereby enabling the biopsy element to approach the lesion from any of at least two different directions, depending on the location of the lesion.

There is further provided, in accordance with an exemplary embodiment of the invention, a system for making x-ray images of a patient's breast, the system comprising:
a) a chair with a back that has an upright position and at least one reclining position;
b) a compression device adapted to hold a breast of a patient seated in the chair in a compressed state, when the chair back is in the upright position or in at least one reclining position; and
c) an x-ray unit capable of being coupled to the chair back so that it is aimed at the breast when the patient is seated in the chair, at least when the chair back is in a reclining position.

Optionally, the x-ray detector is capable of being oriented in any of at least two different angles relative to the breast, thereby enabling the x-ray images to be made from any of at least two different angles depending on the location of the lesion.

Optionally, the x-ray unit is coupled to the back of the chair, such that the x-ray unit remains in substantially the same position and orientation relative to the breast of the patient when the chair goes from the upright position to the at least one reclining positions.

Optionally, the x-ray unit is configured to move to either of two positions in which the x-ray unit directs x-rays at the breast of the patient from different directions, thereby enabling stereotaxic x-ray images of the breast to be made.

Optionally, the angles from which the x-ray images are made covers a range of at least 60 degrees.

Optionally, the patient is leaning back by an angle of at least 10 degrees when the chair is in the reclining position.

Optionally, the angle is at least 30 degrees.

Optionally, the angle is at least 45 degrees.

Optionally, the angle is at least 60 degrees.

Optionally, the angle is at least 75 degrees.

Optionally, the angle is approximately 90 degrees.

There is further provided, according to an exemplary embodiment of the invention, a method of x-ray imaging of a patient's breast, the method comprising:
a) seating the patient in a chair at least the back of which can lean back;
b) compressing the breast of the patient in a compression device;
c) causing the back of the chair to lean back such that the patient is reclining at an angle of at least 10 degrees; and
d) making x-ray images of the compressed breast when the patient is reclining at said angle.

Optionally, compressing the breast in a compression device comprises compressing the breast in a soft compression device as described, before causing the back of the chair to lean back.

In an embodiment of the invention, compressing the breast in a compression device also comprises compressing the breast further with a hard compression device, after compressing the breast in the soft compression device and causing the back of the chair to lean back, and before making the x-ray images, thereby enabling the x-ray images to be of sufficient quality to guide the biopsy.

Optionally, making x-ray images comprises making a stereotaxic pair of x-ray images.

Optionally, the method also includes:
a) coupling the compression device to a biopsy unit; and
b) performing a biopsy of a lesion in the breast, using a biopsy element in the biopsy unit, guided at least by the x-ray images.

Optionally, coupling the compression device to the biopsy unit is done after compressing the breast.

In an embodiment of the invention, the method includes:
a) determining an at least approximate location of the lesion in the breast; and
b) choosing one or more of a direction at which the biopsy element enters the breast, an average direction from which the stereotaxic pair of x-ray images is made, and a direction in which the breast is compressed in the compression device, depending on said location of the lesion.

Optionally, choosing one or more of the directions comprises choosing all of the directions to be approximately the same direction.

Optionally, the method includes:
a) determining whether the lesion is visible in at least one of the x-ray images;
b) if the lesion is not visible, moving the x-ray detector so that its field of view includes a different zone of the breast, and making a new x-ray image;
c) determining if the lesion is visible in the new x-ray image; and
d) repeating (b) and (c) until the lesion is visible in the new x-ray image.

Optionally, moving the x-ray detector comprises moving the x-ray detector together with the biopsy unit, so that the x-ray detector and the biopsy unit maintain the same relative position.

Optionally, the compression device is a compression device as described.

There is further provided, in accordance with an exemplary embodiment of the invention, a method of performing a biopsy on a lesion in the breast, the method comprising:
a) wrapping a flexible band at least part way around the breast;
b) anchoring the flexible band to a base;
c) compressing the breast between the flexible band and the base, by applying tension to the flexible band;
d) coupling one or both of the flexible band and the base to a biopsy unit, with sufficient spatial stability to enable the biopsy unit to perform an x-ray guided biopsy;
e) making x-ray images of the breast while it is compressed between the flexible band and the base; and
f) performing the biopsy on the breast while it is so compressed, using the biopsy unit, guided at least by the x-ray images.

Optionally, wrapping a flexible band at least part way around the breast comprises wrapping the flexible band at least half way around the breast.

Optionally, coupling one or both of the flexible band and the base to the biopsy unit comprises coupling the base to the biopsy unit.

Optionally, the method includes:
a) determining an at least approximate location of the lesion in the breast; and
b) positioning the base on a portion of the breast that depends on said location.

Optionally, performing the biopsy comprises puncturing the flexible band with a biopsy element.

Optionally, the method includes sterilizing a substantial part of the surface of the breast, and the flexible band, before wrapping the flexible band around the breast.

In an exemplary embodiment of the invention, the method includes removing an area of the flexible band from the breast before performing the biopsy, thereby exposing the skin in that area, wherein the biopsy is performed by puncturing said exposed skin by a biopsy element.

Optionally, removing an area of the flexible band from the breast comprises peeling back a strip of the flexible band, starting from an end of the flexible band where it is anchored to the base.

Optionally, performing the biopsy is done while the patient is reclining back by an angle of at least 10 degrees from the vertical. Optionally, the angle is at least 30 degrees, or at least 45 degrees, or at least 60 degrees, or at least 75 degrees, or about 90 degrees.

### BRIEF DESCRIPTION OF THE DRAWINGS

Exemplary non-limiting embodiments of the invention are described in the following sections with reference to the drawings. The drawings are generally not to scale and the same or similar reference numbers are used for the same or related features on different drawings.

FIG. 1 is a side view of a patient seated in a chair, in a generally upright position, with attached x-ray and biopsy units, according to an exemplary embodiment of the invention;

FIG. 2 is a side view of the patient and chair shown in FIG. 1, but in a reclined position;

FIG. 3 is a back view of an embodiment of the chair of the invention shown in FIG. 1;

FIG. 4A is a front view of an embodiment of the chair of the invention shown in FIG. 1;

FIG. 4B is side view of a patient seated in a chair, in an upright position, with x-ray and biopsy units, according to a different exemplary embodiment of the invention;

FIG. 4C is a side view of the patient and chair shown in FIG. 4B, but in a reclined position;

FIG. 5A is a more detailed side view of a biopsy unit, breast support base, compression device, and x-ray unit, according to an exemplary embodiment of the invention;

FIG. 5B is a perspective view of the biopsy unit shown in FIG. 5A, seen from the patient side;

FIG. 5C is a perspective view of the breast support base and compression device shown in FIG. 5A, seen from the side opposite the patient;

FIG. 6A is a schematic vertical projection view of a breast with a lesion, according to an exemplary embodiment of the invention;

FIG. 6B is a side view of a biopsy unit, breast support base, and breast compression device, according to a different embodiment of the invention than that shown in FIG. 5A;

FIG. 6C is a perspective view of the biopsy unit shown in FIG. 6B, seen from the side facing the patient;

FIG. 7 is a front view of a breast in a soft compression device, according to an exemplary embodiment of the invention;

FIG. 8 is a side view of the breast in the soft compression device shown in FIG. 7;

FIG. 9 is a side view of a breast in a soft compression device, according to a different exemplary embodiment of the invention;

FIG. 10 is a perspective side view of a breast in the soft compression device shown in FIG. 9, and showing a biopsy needle positioned for entry into the breast; and

FIG. 11A and FIG. 11B are perspective views of a breast in a soft compression device according to a different exemplary embodiment of the invention, showing a time sequence.

### DETAILED DESCRIPTION OF EXEMPLARY EMBODIMENTS

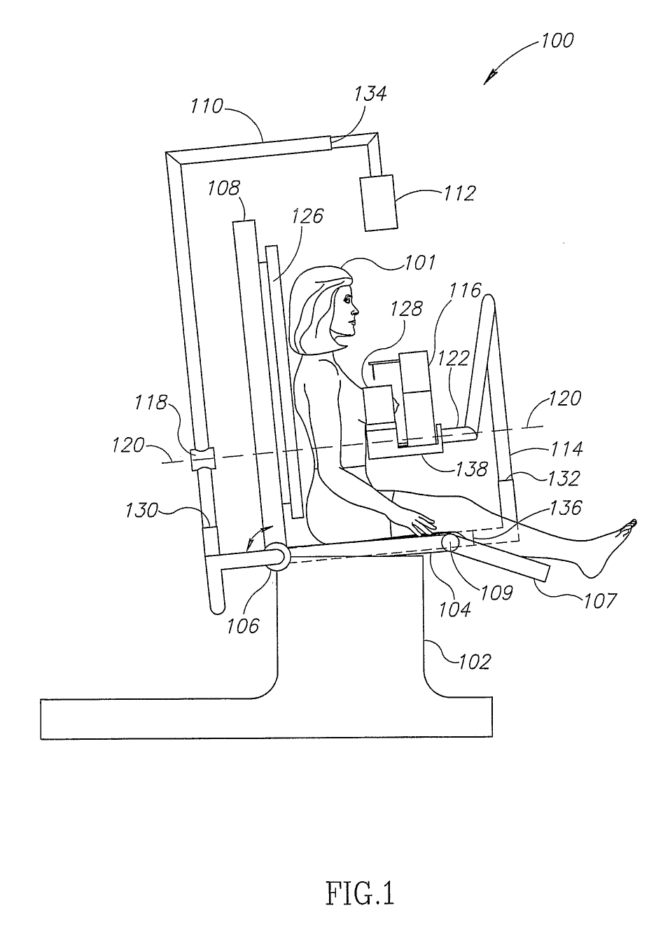

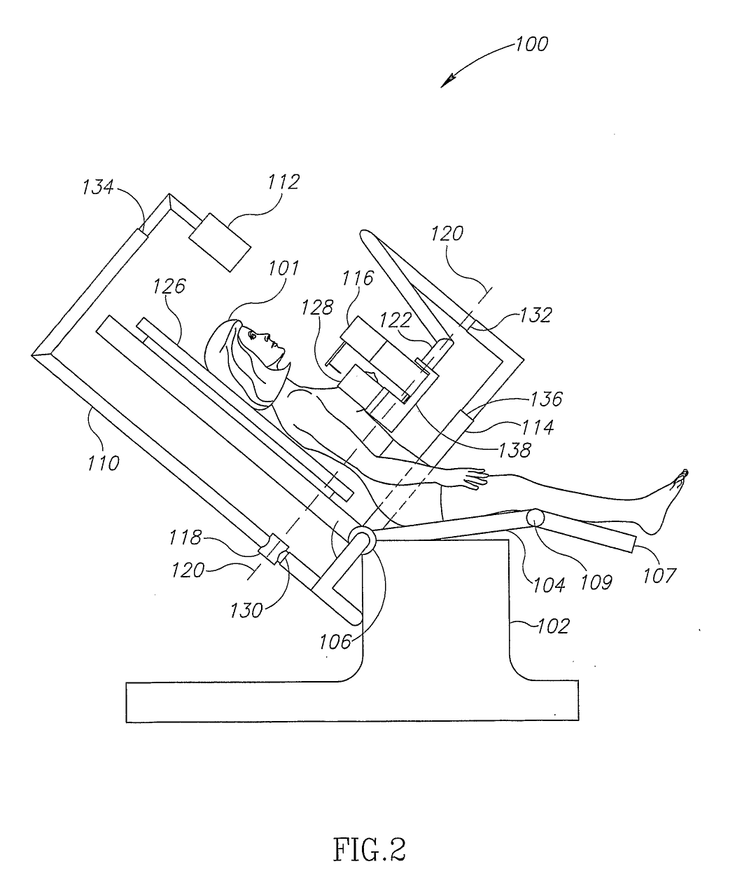

FIGS. 1 and 2 show a patient 101 in a reclining chair 100, in different positions. In FIG. 1, chair 100 is in an upright position. Chair 100 is similar to a dentist's chair, and in fact a commercially available dentist's chair is optionally used, with modifications as will be described. A rotatable joint 106 is supported by a base 102. Seat 104 and back 108 are attached to joint 106, and independently rotate around joint 106. Joint 106 optionally allows the back of the chair to tilt back by any of a continuous range of angles up to, for example, 90 degrees, which would put the patient in a completely supine position. Independently of the back, seat 104 optionally can tilt up by any of a continuous range of angles up to, for example, 10 degrees, or 15 degrees. Alternatively, seat 104 is not free to tilt at all, but remains horizontal, or at a fixed angle, relative to base 102. Alternatively, the angle between seat 104 and back 108 does not change when the chair tilts back.

FIG. 2 shows chair 100 with back 108 tilted by an angle of about 60 degrees, and seat 104 tilted up by about 15 degrees. Optionally, joint 106 allows the back to recline by at least 15 degrees, or at least 30 degrees, or at least 45 degrees, or at least 60 degrees, or at least 75 degrees, or 90 degrees, or even more. Optionally, at the other end of seat 104 from joint 106, there is a leg rest 107 which bends at a joint 109 at the position of the patient's knees, as shown in both FIGS. 1 and 2. Alternatively, joint 109 is supported by its own support base which rests on the floor, or there is no joint 109 and seat 104 is long enough to accommodate the patient's legs.

Optionally, the bottom of base 102 extends well to the back of joint 106, so that the chair will not tip over when it reclines, even with a top-heavy patient sitting in it.

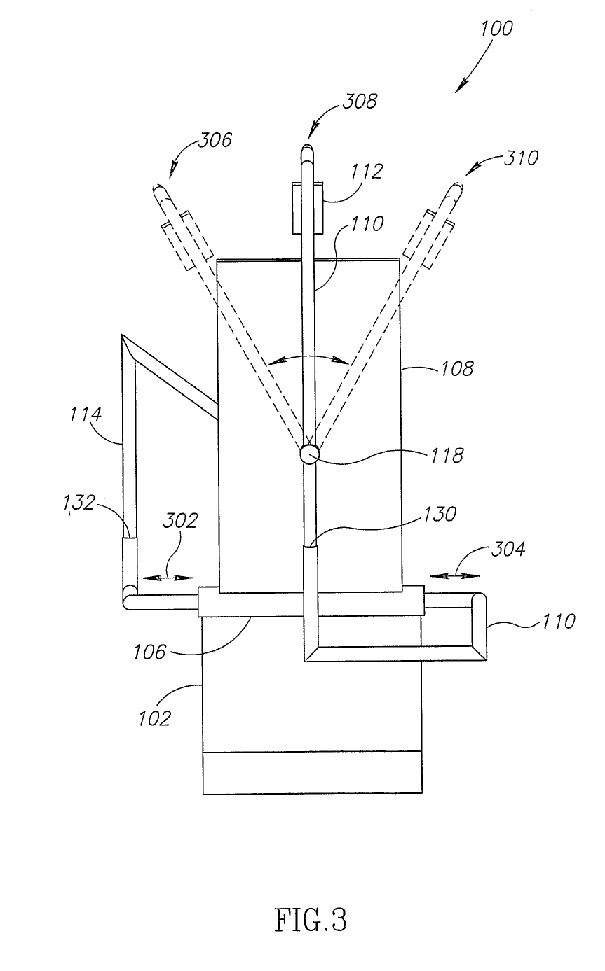

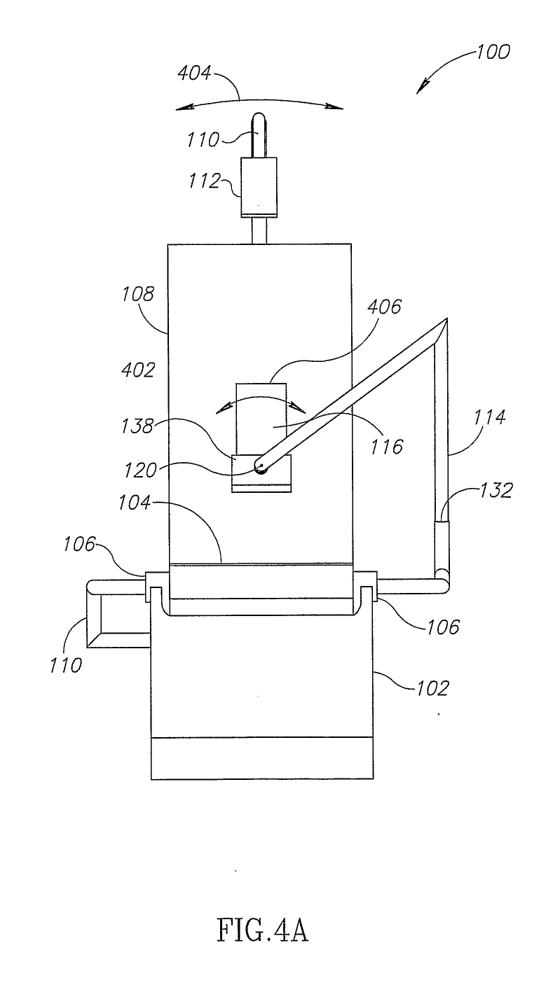

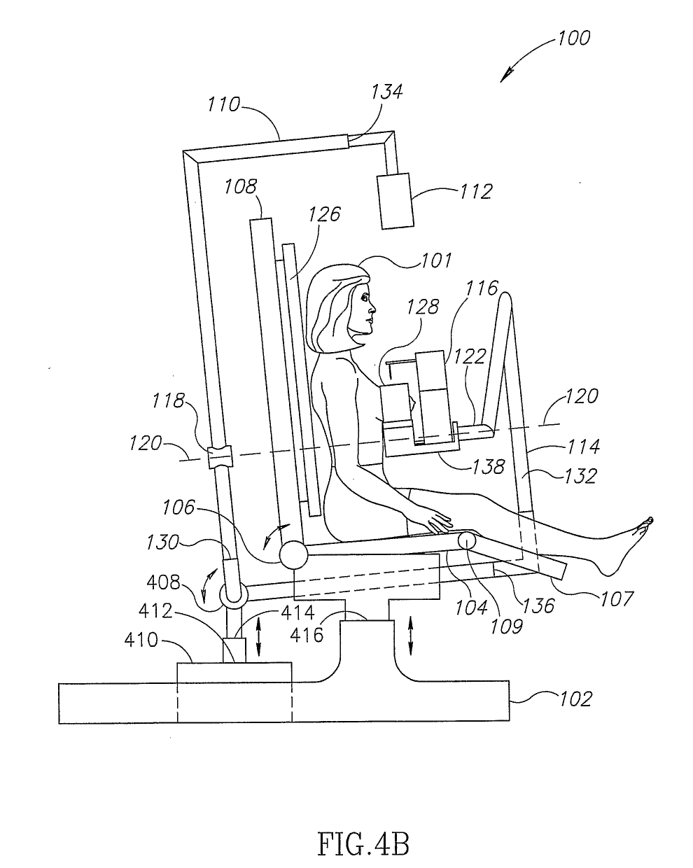

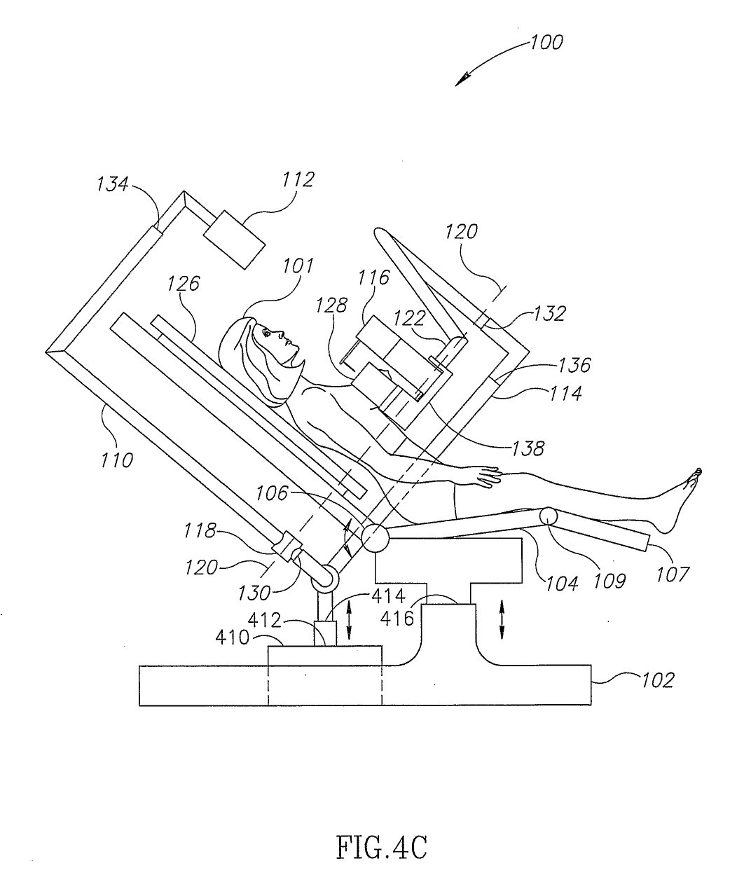

A rigid fixture 110 connects an x-ray unit 112 to the back of the chair, and another rigid fixture 114 connects a support base 138 to the back of the chair. Support base 138 is coupled to a biopsy unit 116, and is also coupled to a breast compression device 128, at least when the breast is being imaged and when the biopsy is performed. When the back of the chair tilts back, both x-ray unit 112 and biopsy unit 116 move together with the back of the chair, maintaining the same position and orientation relative to each other, so that, for example, if the x-ray unit is aligned with a particular location on the biopsy unit, then it will remain aligned with that location when the back of the chair is tilted back, or brought upright. Fixture 110 does, however, have a lockable joint 118, which allows it to bend out of the plane of the drawing in FIG. 1, around an axis 120 which also passes through biopsy unit 116, and in particular through a part 122 of fixture 114 which biopsy unit 116 can rotate around. This bending of fixture 110 and rotating of biopsy unit 116 is illustrated in FIGS. 3 and 4, which are described below, and which show the chair from the back and the front respectively. It should be noted that fixture 110 comprises several segments, whose three-dimensional structure may be better understood by comparing the side view in FIG. 1 to the back view in FIG. 3. Similarly, the three-dimensional structure of fixture 114 may be better understood by comparing the side view in FIG. 1 to the front view in FIG. 4.

Retuning to FIG. 1, back 108 optionally supports a back rest 126, which optionally may be moved forward or backward, either manually or using a motor or actuator, and locked into place at a desired position, in order to position the breast in desired location relative to biopsy unit 116.

To describe the position of the breast or any part of the biopsy unit relative to the back of the chair, we define a Cartesian coordinate system. The z-axis is parallel to the back of the chair, and is vertical when the back of the chair is upright, as in FIG. 1. The x-axis is normal to the back of the chair, and the y-axis extends in the left and right directions of the patient, normal to the plane of the drawing in FIGS. 1 and 2.

It is noted that the z-coordinate of the breast of the patient depends on the patient's height. Furthermore, even for a given patient, the z-coordinate of the breast depends on the angle between the back and seat of the chair, if the patient keeps her knee over joint 107 of the chair, since the patient's hip joint does not coincide with the axis of joint 106 of the chair. For both these reasons, the z-coordinate of the biopsy unit is optionally adjustable. For example, fixture 114, which supports the biopsy unit, optionally has a telescoping joint 132, by which the z-coordinate of the biopsy unit can be adjusted. Optionally, the z-coordinate of x-ray unit 112 is also adjustable, through telescoping joint 130 of fixture 110. Comparing FIGS. 1 and 2, it is apparent that the x-ray unit and biopsy unit have been brought down, by adjusting telescoping joints 130 and 132, in order to keep the x-ray unit and the biopsy unit in the same relative position to the breast, when the back of the seat reclines.

Alternatively, when the back of the chair first reclines, seat 104 is tilted up by the same angle, so that the patient's hip joints remain bent at the same angle, keeping the patient's breast at approximately the same z-coordinate as the back of the chair reclines. When the back of the chair and the seat are both tilted by a large angle, for example 45 degrees or 60 degrees, so that the weight of the patient is mostly supported by the back of the chair rather than by the seat, then the seat is tilted back down at least part of the way, allowing the patient to stretch out comfortably. Although the patient's knees will now no longer be situated over joint 109, which may not affect her comfort if joint 109 is nearly unbent, her breast will remain at approximately the same z-coordinate as when she was sitting upright, making it unnecessary to make large adjustments in the z-coordinate of the biopsy unit. Optionally, the back is now further reclined if desired, and her breast will tend to remain at the same z-coordinate, since most of her weight is supported by the back of the chair.

Alternatively, instead of joint 106 being a simple joint as shown in FIGS. 1-4, a more complicated design is used in which joint 106 rotates around an axis that coincides with, or is closer to, the patient's hip joint. In this case, the z-coordinate of the patient's breast would tend not to change so much, if at all, when the patient reclines, although there might be gap between the back and seat of the chair when the back of the chair is upright.

Telescoping joints 130 and 132 need not be at the positions shown, but are optionally located on any vertical portion of fixtures 110 and 114 respectively. Optionally, fixtures 110 and 114 are mechanically linked in some way, so that when the z-coordinate of the biopsy unit is changed, the z-coordinate of the x-ray unit is changed by the same amount, keeping the x-ray unit in the same relative position to the biopsy unit. Alternatively, telescoping joints 130 and 132 are each controlled by motors which are controlled to always move the x-ray unit and the biopsy unit by the same distance in z. Alternatively, the x-ray unit and biopsy unit are manually adjusted to keep the same relative positions, when they are moved in z.

Optionally, instead of or in addition to using back rest 126 to adjust the x-coordinate of the breast relative to biopsy unit 116, there is a telescoping joint 136 on fixture 114, which adjusts the x-coordinate of the biopsy unit, to bring it into a proper position relative to the breast. In this case at least, optionally there is no back rest 126. Optionally, there is also a telescoping joint 134 on fixture 110, to adjust the x-coordinate of x-ray unit 112, so that the x-ray unit remains at the same position relative to the biopsy unit, when the x-coordinate of the biopsy unit is adjusted. Optionally, joints 134 and 136 are located at any other locations on fixtures 110 and 114, respectively, where fixtures 110 and 114 are oriented in the x-direction. Optionally, fixtures 110 and 114 are mechanically linked so that the x-ray unit and biopsy unit always move the same distance in x when their positions are adjusted, or there are motors which are controlled to always move the x-ray unit and the biopsy unit the same distance in x, or the x-ray unit and the biopsy unit are manually adjusted to always have the same relative position when they are moved in x.

Similar features for adjusting the y-coordinates of the x-ray unit and biopsy unit will be described below, with reference to FIG. 3. Alternatively or additionally, the patient moves over sideways in the seat so that her breast has the proper y-coordinate relative to the biopsy unit and x-ray unit.

Optionally, the patient's breast is put into compression device 128 when the patient is sitting upright, and the back of the chair is then moved back until the patient is reclining at a comfortable angle. This is particularly useful for patients with small and/or sagging breasts, which are difficult or impossible to capture in a compression device when the patient is reclining. For patients with larger breasts, it may be possible for the patient to recline first, at least up to a certain angle, and then to put the breast into compression device 128.

Optionally, compression device 128 is detachable from biopsy unit 116, for example if compression device 128 is the soft compression device shown in FIGS. 1 and 2, and described in detail in FIG. 7. If compression device 128 is detachable, then optionally the breast is put into compression device 128 when it is detached from biopsy unit 116. The compression device optionally remains detached from the biopsy unit as the back of the chair reclines, or the compression device is loosely attached to the biopsy unit, but not locked into place, as the back of the chair reclines. Once the patient is reclining at a comfortable angle, the x, y and z coordinates of biopsy unit 116 are adjusted, and/or the patient adjusts her position, so that the compression device 128 can be attached to the biopsy unit and locked into place.

Alternatively, in particular if the compression device is permanently attached to biopsy unit 116, for example, if the compression device is a conventional compression device with a polycarbonate compression plate, then the breast is put into the compression device when the compression device is attached to the biopsy unit, whose coordinates are adjusted initially to allow the breast to be put into the compression device. If this is done when the patient is sitting upright, then, as the back of the seat reclines, the coordinates of the biopsy unit, particularly the z-coordinate, are adjusted, continuously or frequently, so that the biopsy unit remains in nearly the same position relative to the patient, avoiding any strain on the breast which would cause discomfort, or would cause the breast to slip out of the compression device.

In any of these cases, the x-ray unit optionally remains at the same position relative to the biopsy unit, when the back of the chair reclines and when the coordinates of the biopsy unit are changed, as described above. Alternatively, the x-ray unit is adjusted to be at a proper position relative to the biopsy unit only later, any time before x-ray images are made.

FIG. 3 shows a rear view of chair 100, when the back is in an upright position. Fixture 110 is attached to one end of joint 106, to a part of joint 106 that moves rigidly with the back of the chair, when the back moves into a reclining position, and fixture 114 is attached to the other end of joint 106, also moving rigidly with the back of the chair, although part of fixture 114 and all of biopsy unit 116 are hidden by the back of the chair in FIG. 3. As long as fixtures 110 and 114 move rigidly with the back of the chair, they may be attached in any convenient way that does not get in the way of the patient. The particular configuration shown in FIGS. 1-4A is merely illustrative.

Optionally, however, fixtures 110 and 114 are free to move together to the left and right with respect to the back of the chair, as shown by arrows 302 and 304, in order to position the biopsy unit laterally (in the y-direction) with respect to the patient's breast, and may be locked into place once the biopsy unit is correctly positioned. Alternatively or additionally, as noted above, the patient moves left or right, in order to position the breast properly with respect to the biopsy unit, prior to immobilizing the breast with respect to the biopsy unit.

Alternatively, x-ray unit 112 is not attached to the chair, but is a conventional moveable x-ray unit, similar to a dental x-ray machine, which is brought into a proper position and orientation once the patient is in the reclining position and ready for the biopsy. Optionally, in this case, the x-ray unit is attachable to the chair in a way that gives it a known position and orientation relative to the chair, particularly to the back of the chair. A potential advantage of having the x-ray unit rigidly attached or attachable to the chair, as in FIGS. 1-4A, is that the stereotaxic images may be more accurate, since the precise position of the x-ray unit relative to the biopsy unit, and in particular relative to the x-ray detector, is known. Although the x-ray unit still changes its angle, relative to the biopsy unit, between making the first and second image, it only has one degree of freedom if it is attached to the chair, and even the uncertainty in this angle can be avoided by configuring fixture 110 so that the x-ray unit locks into place at prescribed angles. Furthermore, if the x-ray unit is attached rigidly to the chair, it will already be in the correct position when the patient is reclining and ready for the biopsy, saving time.

Alternatively or additionally, the support base with the biopsy unit is not attached to the chair, but is brought into place and attached to compression device 128 once the patient is in the reclining position. Optionally the support base is also locked onto the chair then, optionally in such a way that it has a known position and orientation relative to the chair, particularly to the back of the chair. A potential advantage of having the support base and biopsy unit permanently attached to the chair is that the biopsy unit will be at least in approximately the correct position when the patient is reclining and ready for the biopsy, saving time. Furthermore, if the support base with the biopsy unit moves together with the back of the chair when the back of the chair is reclined, or can be readily attached to the chair in a same position and orientation relative to the back of the chair, then a biopsy may be readily performed with the patient reclining at any angle. Different patients may prefer reclining at different angles, or the same patient may prefer different angles at different times.

A potential advantage to having the support base with the biopsy unit locked to the chair at least during the biopsy is that the biopsy unit will not inadvertently move relative to the chair, pulling at the compression device to which it is attached, and causing discomfort to the patient, or causing the breast to move relative to the biopsy unit, and compromising the accuracy of the biopsy.

Joint 118 is located in fixture 110, and allows x-ray unit 112 to swing to the left and right around joint 118. For example, x-ray unit 112 may swing to the left, into position 306, or straight above the chair into position 308, or to the right into position 310. There are two reasons for allowing x-ray unit 112 to swing around joint 118. First, in order to find the three-dimensional location of a lesion, at least two x-ray images are obtained from different angles, as in conventional stereotaxic x-ray biopsy systems. Typically, images are taken with the x-rays coming from an angle of 15 degrees on each side of a zero angle. Second, the optimal angle for making x-ray images of the breast, and for inserting the biopsy needle, may vary depending on the location of the lesion. For example, the biopsy needle is optionally inserted at an angle that will minimize the distance it must go into the breast to reach the lesion, and the zero angle for the x-ray unit is adjusted to match the angle of the biopsy unit, as described below in the description of FIG. 4A.

Optionally, the zero angle of x-ray unit 112, and the angle of insertion of the biopsy needle, can change over a range of at least 30 degrees in each direction from the vertical, or at least 60 degrees in each direction from the vertical, or at least 90 degrees in each direction from the vertical, or 135 degrees in each direction from the vertical, or less than 30 degrees, or more than 135 degrees.

Alternatively, the zero angle of the x-ray unit does not change at all, and the angle of insertion of the biopsy needle does not change, but the x-ray unit swings to the left and right to make stereotaxic pairs of x-ray images. The maximum swing angle for the x-ray unit between stereotaxic pairs, in addition to its maximum change in zero angle, is optionally 15 degrees on each side of the zero angle, or 20 degrees, or 30 degrees, or more than 30 degrees, or less than 15 degrees.

Optionally, joint 118 is locked, so that x-ray unit 112 cannot swing back and forth, once x-ray unit 112 is in a desired position. Optionally, there is a sensor, not shown in FIG. 3, which senses the position of x-ray unit 112, for example by sensing the angle of joint 118, and a sensor which senses the orientation of the support base with the biopsy unit. A potential advantage of having such sensors is that they can be used to verify that the zero angle of the x-ray unit is the same as the angle of orientation of the biopsy unit, or to measure any discrepancy in the angles. When the position of the x-ray source is precisely known, the stereotaxic x-ray images can be used to guide the biopsy needle automatically or semi-automatically, first calculating the precise three-dimensional location of the lesion relative to the biopsy unit. Optionally, this calculation takes into account any discrepancy between the zero angle of the x-ray unit and the angle of orientation of the biopsy unit. Optionally, there are a finite number of discrete angles at which joint 118 may be locked, in which case a relatively simple sensor may be used to sense the position of x-ray unit 112. Alternatively or additionally, joint 118 may be locked at any of a continuous range of angles. The same options apply to the orientation of the support base with the biopsy unit.

FIG. 4A is a front view of chair 100, in the upright position (without the patient). For clarity, leg rest 107 and joint 109 are not shown. Fixture 114 is shown, with one end attached to one side of joint 106. Support base 138 is attached to the other end of fixture 114, and biopsy unit 116 is mounted on support base 138. X-ray unit 112 is attached to fixture 110, which is partly hidden behind the back of the chair. Support base 138 is mounted on fixture 114 in such a way that it can rotate around axis 120 (visible only as a single point in FIG. 4A, since it is perpendicular to the plane of the drawing), as shown by arrow 402. As may be seen in FIG. 1, axis 120 also passes through joint 118. When support base 138 with biopsy unit 116 rotates in one direction or the other around axis 120, x-ray unit 112 optionally rotates by the same angle around joint 118, as shown by arrow 404, so that the zero angle of x-ray unit 112 is always aimed toward a part 406 of biopsy unit 116 which is on the top in FIG. 4A.

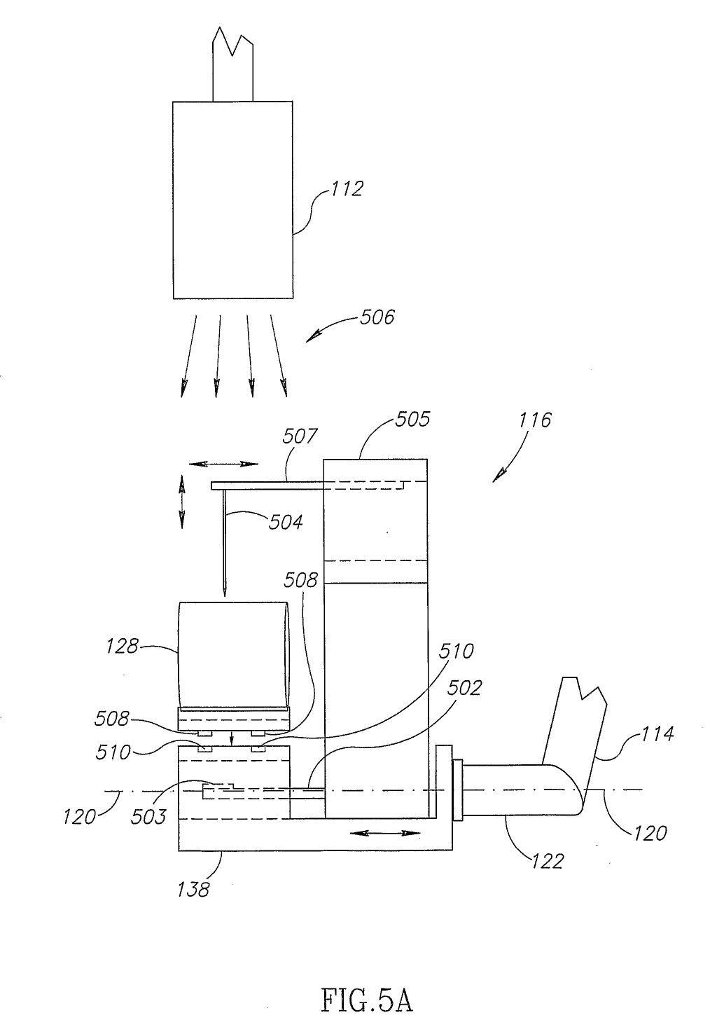

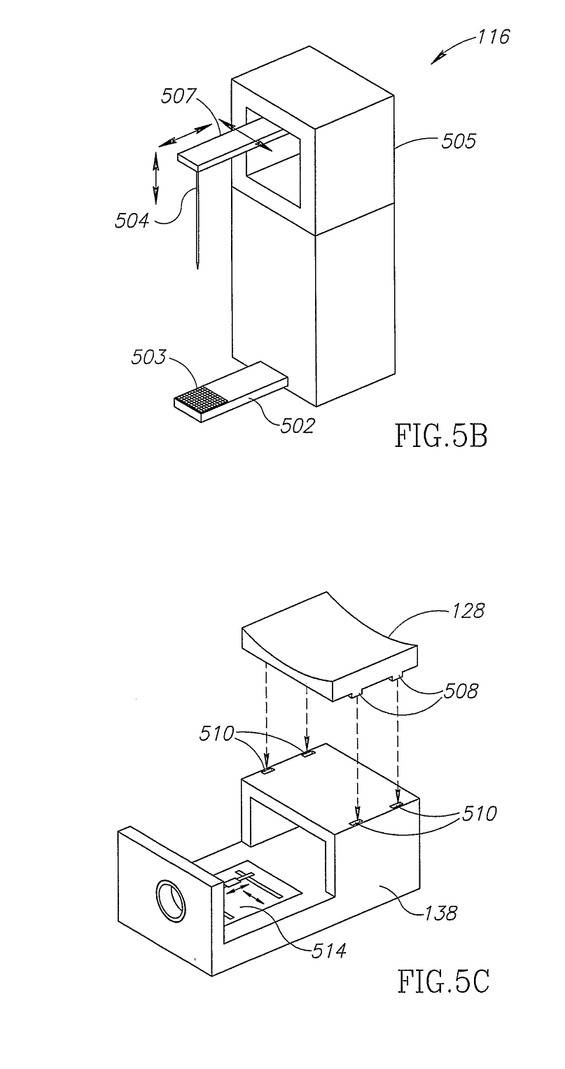

In particular, biopsy unit 116 includes a holder 502 for an x-ray detector, for example a solid-state detector array or a holder for x-ray film, shown in FIG. 5A, and biopsy unit 116 optionally rotates about axis 120 so that the normal to the plane of the x-ray detector is always oriented toward x-ray unit 112, when x-ray unit 112 is at its zero angle. Also, biopsy unit 116 includes a biopsy needle 504, also shown in FIGS. 5A and 5B, which optionally remains at the same position and orientation relative to the zero angle of the x-ray beam, when x-ray unit 112 swings around joint 118 to change its zero angle, and support base 138 with biopsy unit 116 rotates around axis 120. Hence, for example, a control system which uses the x-ray images to calculate the proper position, direction and distance of insertion of the biopsy needle, will work in the same way regardless of the orientation of biopsy unit 116 relative to the chair.

Support base 138 optionally does not rotate about axis 120 when x-ray unit 112 swings around joint 118 to take a pair of stereotaxic x-ray images from different angles, since the breast compression device is firmly locked to support base 138 at this time. For this reason, axis 120 optionally passes through x-ray detector holder 502, as shown in FIG. 5A, so that both images are located at the same place on the x-ray detector, or the x-ray detector is wide enough so that both images fit on it without moving it. Support base 138 with biopsy unit 116 does rotate about axis 120, and the zero angle of x-ray unit 112 optionally rotates by the same angle about joint 118 (located on and aligned with axis 120), when it is desired to perform a biopsy from a different angle.

The physician performing the biopsy decides on the best angle for the biopsy needle to approach the breast, based on the location of the lesion as indicated by a mammogram, for example. Optionally when the patient is still sitting in an upright position, the support base with the biopsy unit is rotated to this angle, and the breast is compressed from this angle, or approximately this angle, by the compression device. If the compression device is not coupled to the support base when the breast is compressed, then optionally the support base is not rotated to this angle until a later time, before the compression device is coupled to the support base. Alternatively, the support base is rotated to this angle even before the patient is seated in the chair.

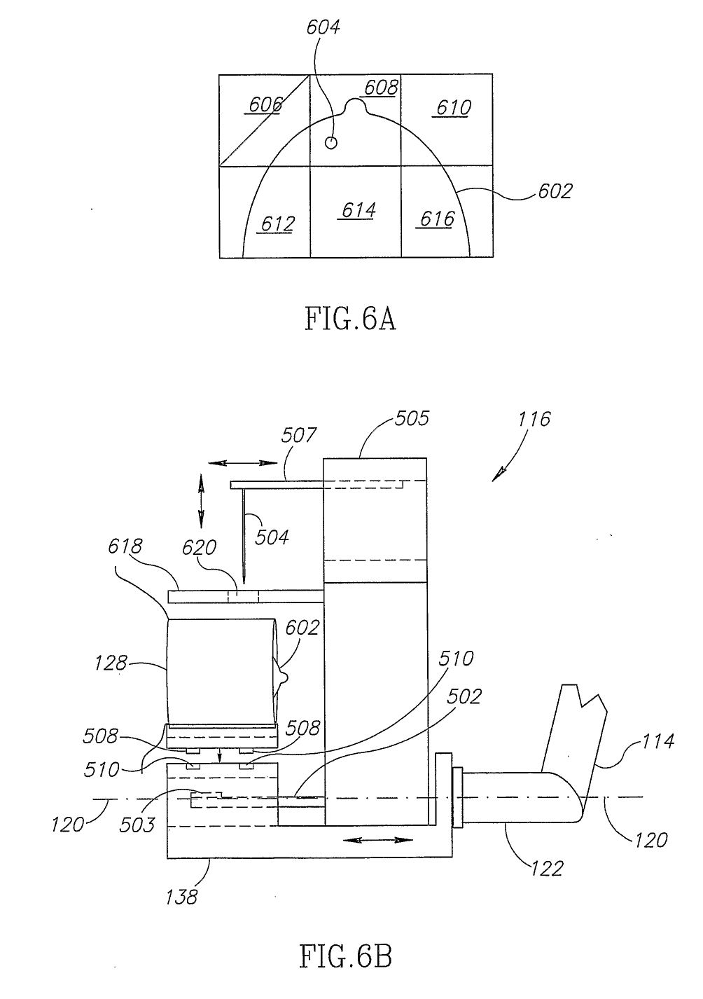

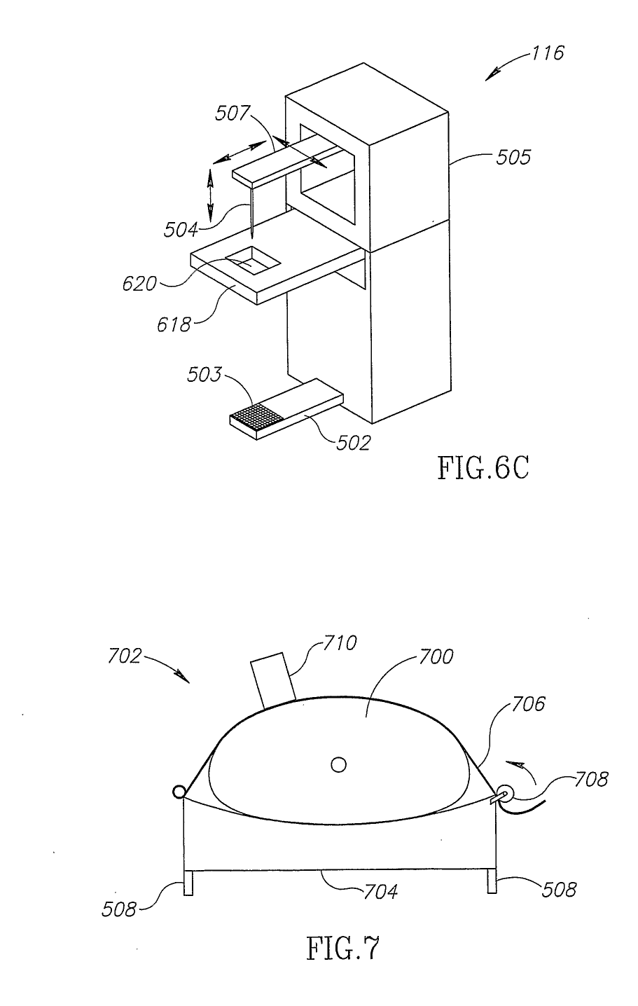

Once the breast is compressed, the back of the chair is then reclined, if the patient was sitting upright when the breast was compressed, and the compression device is coupled to the support base, if it wasn't already coupled to the support base. If necessary, the position of the support base in the x, y, and z direction is adjusted, and/or the position of the patient is adjusted, before and/or after the back of the chair is reclined, in order to bring the support base and the compression device into a position where they can be coupled together. Optionally, as will be explained below when describing FIG. 6, after locking the compression to the support unit, a test x-ray image is made, for example with x-ray unit 112 at its zero angle, to make sure that the x-ray detector is positioned with respect to the breast so that the lesion is visible in the x-ray image, and adjustments in the position of the biopsy unit relative to the support base are made if necessary.

The zero angle of the x-ray unit is rotated to the same angle as the biopsy unit, optionally at the same time as the biopsy unit is rotated, or at any time before x-ray images are made. When a stereotaxic pair of x-ray images is made, the x-ray unit is rotated first 15 degrees, for example, in one direction, then 15 degrees, for example, in the other direction, from its zero angle. Alternatively, an angle greater than or less than 15 degrees is used when making stereotaxic images.

FIGS. 4B and 4C show an alternative configuration for mounting fixtures 110 and 114, including x-ray unit 112 and biopsy unit 116. Instead of attaching fixtures 110 and 114 to the back of the chair through joint 106, as in FIGS. 1-4A, fixtures 110 and 114 are mounted on a separate joint 408, which is mounted on a stand 410 which rests on the floor. Joint 408 is located close to joint 106.

Stand 410 remains fixed relative to base 102 of the chair, for example by anchoring both stand 410 and base 102 to the floor, or by anchoring stand 410 to base 102. When the chair leans back by tilting the back of the chair around joint 106, fixtures 110 and 114 tilt back by the same angle around joint 408. FIG. 4B shows fixture 110 and chair back 108 both in an upright position, and FIG. 4C shows them both tilted back by about 60 degrees.

Optionally, the tilting of chair back 108 is synchronized with the tilting of fixtures 110 and 114. For example, chair back 108 and fixtures 110 and 114 are controlled by motors which are controlled to always tilt the chair back and the fixtures by the same angle. Alternatively, there is a sensor, located for example in joint 106, which measures the tilting angle of chair back 108, and there is a motor, located for example in joint 408, which is controlled to tilt fixtures 110 and 114 by the angle detected by the sensor. Alternatively, the sensor senses the angle of the fixtures, and the motor controls the angle of the chair back. Alternatively, both angles are adjusted manually to be the same.

Because joint 408 is located close to joint 106, x-ray unit 112 and biopsy unit 116 remain approximately in the same position, relative to the back of the chair, when the back of the chair is tilted back. Because joint 408 is not exactly co-axial with joint 106, the relative positions do not remain exactly the same. To compensate for changes in the relative positions of the x-ray and biopsy units to the back of the chair when the chair leans back, telescoping joints 130, 132, 134 and 136 are optionally used to adjust the positions of x-ray unit 112 and biopsy unit 116 in x and z. Such adjustments are optionally made in any event to compensate for changes in the position of the patient's breast relative to the back of the chair.

Stand 410 optionally has a mechanism 412 which allows joint 408 to move in the y direction relative to the chair. For example, there is a track at the bottom of stand 410, and the upper part of stand 410, together with joint 408 and fixtures 110 and 114, moves back and forth in the track. Any other kind of mechanism producing linear motion is optionally used, and mechanism 412 need not be located near the bottom of stand 410, as shown in FIGS. 4B and 4C, but is optionally located higher up in stand 410, for example adjacent to joint 408. Optionally, fixtures 110 and 114 do not move in the y direction relative to joint 408, as fixtures 110 and 114 in FIG. 3 move in the y direction relative to joint 106, but instead the y position of the x-ray unit and biopsy unit are adjusted by using mechanism 412. Such adjustment in the y position is used, for example, to move the biopsy unit from one breast to the other breast.

Stand 410 optionally has a lift mechanism 414 which raises and lowers joint 408, together with fixtures 110 and 114. Base 102 of the chair optionally has a lift mechanism 416, which raises and lowers the chair, similar to controls usually found on dentists' chairs. Optionally, mechanism 414 and mechanism 416 are controlled to move together. When the doctor raises or lowers the chair, for example to put the patient at a more comfortable height for the doctor to work with, then fixtures 110 and 114, together with x-ray unit 112 and biopsy unit 116, are raised or lowered by the same distance, keeping the x-ray unit and biopsy unit in the same position relative to the chair. Any of the methods mentioned previously, for controlling joint 408 and joint 106 to tilt by the same angle, are optionally used to control mechanism 414 and mechanism 416 to raise or lower the chair and joint 408 by the same distance.

Optionally, a configuration similar to that shown in FIGS. 4B and 4C is used for making x-ray images of the breast for purposes other than guiding a biopsy, for example for diagnosis. In this case, optionally there is no fixture 114 or biopsy unit 116, but joint 408 supports only fixture 110 and x-ray unit 112. Optionally the breast is compressed by a soft compression device which is not attached to anything. Alternatively, there is a fixture 114 to which a breast compression device is attached, for example a conventional flat plate compression device, which may be better able to exert the larger compressive forces generally used for diagnostic x-ray images of the breast. In either case, an x-ray detector is coupled in a spatially stable way to the compression device.

FIG. 5A is a more detailed side view of biopsy unit 116, of either FIG. 1 or FIG. 4B. To describe FIGS. 5A, 5B, and 5C, we define new Cartesian coordinates X, Y, and Z, oriented with respect to the biopsy unit, which, as noted previously, can rotate around axis 120. Since axis 120 is in the x-direction, the X-direction is the same as the x-direction, parallel to axis 120. The Z-direction is the vertical direction in FIG. 5A, and the Y-direction is normal to the plane of the drawing in FIG. 5A.

X-ray detector holder 502 is rigidly attached to, and extends to the left of, biopsy unit 116, near the bottom. “Bottom” and “left” here, as well as “above” elsewhere in the description of FIG. 5A, refer to the orientation of biopsy unit 116 as shown in FIG. 5A, and biopsy unit 116 need not be oriented in the same way when it is used; in fact, as noted above, it may rotate into different orientations around axis 120, and its orientation also changes when the chair reclines. Optionally, x-ray detector holder 502 holds a digital x-ray detector array 503, typically a 50 mm by 50 mm square in the x and y directions, located at the left end. Alternatively, x-ray film is used for detecting x-rays, and x-rays can be detected over a larger area than 50 mm by 50 mm.

A potential advantage of using a digital x-ray detector is that the image can be automatically sent, in digital form, to a controller 505, optionally located in the biopsy unit, which controls a biopsy needle 504, using a robot arm 507 which optionally moves in three dimensions. The controller optionally comprises a computer, and optionally calculates the three-dimensional location of the lesion from the stereotaxic pair of x-ray images, optionally with input from a physician who indicates the location of the lesion on a screen by clicking on it with a mouse for example, and the controller then directs the biopsy needle to the lesion, optionally with little or no further human intervention. This can potentially be done much more rapidly using a digital x-ray detector, than using x-ray film, which has to be developed, and scanned, or the location of the lesion in the stereotaxic images has to be measured manually. Manual measurement of the position of the lesion in the images is also possible with a digital detector, for example to verify that the control software is working properly, or to debug it. Optionally, with either a digital detector or x-ray film, the three-dimensional position of the lesion is calculated by the physician from the measured position of the lesion in the stereotaxic images, and the result is input into the controller.

Control system 505 need not physically reside entirely within biopsy unit 116, as shown in FIG. 5A, but could rely in part on a remotely located computer, for example, in communication with biopsy unit 116.

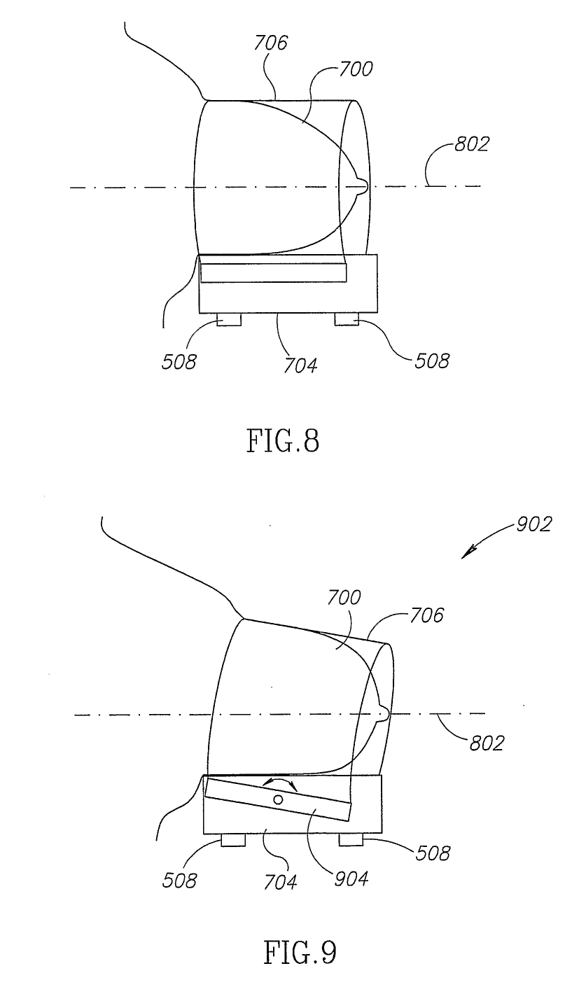

In an embodiment of the invention, biopsy unit 116 is coupled to and above support base 138, and x-ray detector holder 502 optionally extends into a hollow space inside support base 138. Breast compression device 128 has a coupling mechanism 508, which couples to a receptor 510 above the hollow space in the support base, so that the breast is situated above the x-ray detector holder. In FIG. 5A, as well as in FIG. 5C, coupling mechanism 508 is shown schematically as a set of tabs which fit into receptor 510, drawn as a set of slots. However, any coupling mechanism known to the art, which allows compression device 128 to be locked rigidly to support base 138, is optionally used. Although compression device 128 is drawn to look like the soft compression device shown in FIGS. 7 and 8, optionally any breast compression device is used, including any compression device known in the art, or the soft compression device shown in FIG. 9. A conventional flat plate compression device is optionally attached to support base 138 in a different way than shown in FIG. 5A for soft compression device 128, for example it is permanently attached. Alternatively, a conventional flat plate compression device is attached directly to biopsy unit 116.

X-rays 506 from x-ray unit 112 pass through the compressed breast in compression device 128, and reach x-ray detector array 503 where they produce an image. Because axis 120 passes right through x-ray detector holder 502, and specifically through digital array 503, the image always falls substantially centered on digital array 503, regardless of the angle at which x-ray unit 112 is rotated relative to biopsy unit 116. In particular, both stereotaxic images will fall on digital array 503. If x-ray film is used instead of a digital detector, then optionally the x-ray film is located some distance off axis 120, so that the two stereotaxic images will not overlap, but will be located side by side on the x-ray film. X-ray images, for example single images or stereotaxic pairs, are also optionally made during the biopsy, as the needle approaches the lesion, to make sure the needle is positioned correctly. Alternatively or additionally, ultrasound images are used for final positioning.

Biopsy unit 116 is coupled to support base 138 in a way that allows it to be locked into place, so biopsy unit 116 does not move relative support base 138 (and hence relative to the breast), once the x-ray images have been made. Optionally, the coupling between biopsy unit 116 and support base 138 allows them to move relative to each other, in the X and Y directions, before being locked into place. As will be explained below in describing FIG. 6, this allows x-ray detector 503, which is smaller than the breast, to move into a position under the lesion, so the lesion will be visible in the x-ray images. An XY positioner 514, shown schematically in FIG. 5C, allows biopsy unit 116 to move in the X or Y direction relative to support base 138, until it is locked into one position.

Alternatively, particularly if digital array 503 is large enough to image the entire breast, or if x-ray film is used for the x-ray detector, there is no XY positioner, and biopsy unit 116 is rigidly attached to support base 138. Optionally biopsy unit 116 is also rigidly attached to support base 138, without an XY positioner, if a conventional flat plate compression device is used, attached either to biopsy unit 116 or to support base 138. This is particularly true if, as is usual in conventional flat plate compression devices, there is only a single 50 mm by 50 mm window in the flat plate, and the biopsy can only be performed through that window. Then there is no reason to move the biopsy unit relative to the breast, once the breast is compressed, and if the lesion is not located in the window, then the breast is released from compression, moved to a different position relative to the window, and recompressed.

If biopsy unit 116 is attached rigidly to support base 138 without an XY positioner, then fixture 114 is optionally attached directly to biopsy unit 116, instead of to support base 138, and support base 138 need not extend under biopsy unit 116, but is optionally attached to the left side of biopsy unit 116, under the compression device and above the x-ray detector. Alternatively, in this case, there is no separate support base 138, but a bottom portion of the breast compression device, whether a soft compression device or a flat plate compression device, is attached to biopsy unit 116 on the left side, or is a permanent part of biopsy unit 116, above the x-ray detector.

FIG. 5B shows a perspective view of biopsy unit 116, without support base 138 or compression device 128, but including x-ray detector holder 502 and digital array 503. Robot arm 507, with attached biopsy needle 504, is free to move in the X, Y, and Z directions, as shown by the three two-headed arrows, but the biopsy needle preferably does not rotate. The needle is preferably always oriented in the Z direction. If it is desired to perform a biopsy by approaching the breast from another angle to the left or right, but still parallel to the chest wall of the patient, then, as noted previously, the entire support base with the biopsy unit is rotated about axis 120. X-ray guided biopsies are generally only done with the needle oriented parallel, or nearly parallel, to the chest wall of the patient, to avoid any danger of puncturing the heart, and biopsy unit 116 as shown in FIG. 5B only does biopsies with the needle parallel to the chest wall. Optionally, robot arm 507 has the ability to tilt up to some maximum angle away from being parallel to the chest wall, to allow the needle to reach lesions that are close to the chest wall.

FIG. 5C is a perspective view of support base 138, including a schematically drawn XY positioner 514, and a portion of compression device 128 which is locked to support base 138. For clarity, the portion of compression device 128 which wraps around the breast is not shown in FIG. 5C. The bottom of biopsy unit 116, not shown in FIG. 5C, is attached to XY positioner 514, mounted on support base 138, allowing biopsy unit 116 to move in the X and Y directions relative to support base 138. This is done in order to put the lesion into the field of view of the x-ray detector, as explained below in FIG. 6A. Once the biopsy unit is in a desired position in X and Y, the XY positioner is locked in place, and the biopsy unit can no longer move with respect to the breast. Optionally, XY positioner uses a motor to move biopsy unit 116 in X and/or Y. Alternatively or additionally, XY positioner allows biopsy unit 116 to be moved manually. Optionally, there are discrete positions in X and/or Y into which the XY positioner and biopsy unit can be locked. For example, there are a plurality of holes at different positions on the bottom of the biopsy unit, and one hole in the bottom of the support base, under the biopsy unit, and a peg goes through the hole in the support base and one of the holes in the biopsy unit, fixing the biopsy unit in place. Alternatively, the XY positioner and biopsy unit can be locked into place at any of a continuous range of positions, in X or in Y or both.

The parts in all the drawings are necessarily drawn to scale, but may be bigger or smaller than shown in one or more of their dimensions. In particular, digital array 503 may be bigger than shown in FIG. 5B, and XY positioner 514 may be bigger than shown in FIG. 5C.

It should be noted that, because the support base tilts when the back of the chair is tilted, and also tilts when it is rotated around axis 120, the biopsy unit is not always directly above the XY positioner and the support base, with respect to gravity. Depending on the range of reclining angles of the chair, and the range of zero angles for the x-ray unit (which corresponds to the rotation angle of support base 138 around axis 120), support base 138 may be tilted up to some maximum angle, with respect to gravity, and may even be partially or completely turned upside down. Hence, the mounting of biopsy unit 116 to XY positioner 514 and to support base 138 is such that biopsy unit will not fall off under the influence of gravity, even up to the maximum tilt angle. XY positioner 514 need not resemble the XY positioner schematically drawn in FIG. 5C, but any kind of XY positioner known to the art is optionally used.

As noted previously, detector array 503 is typically a 50 mm by 50 mm square, too small to image the entire breast at once (although larger arrays can be used, optionally without an XY positioner). Robot arm 507 typically has a range of motion of 50 mm in X and 50 mm in Y, with the range of biopsy needle 504 located directly over detector array 503. Digital x-ray detectors with much larger arrays, and robot arms with much large range, can be expensive. Hence detector array 503 needs to be positioned under a part of the breast where the lesion is located. FIG. 6A schematically shows an x-ray image of a breast 602, for example a conventional mammogram made with x-ray film, with a lesion 604. This image is made, for example, with x-rays coming from above the breast. Superimposed on the image are a set of six zones, 606, 608, 610, 612, 614, and 616, arranged in a 3 by 2 rectangular grid. Alternatively a different number or arrangement of zones is used. Depending on which zone the lesion is located in, the physician uses XY positioner 514 to position biopsy unit 116, and hence x-ray detector array 503, in one of a set of discrete positions relative to support base 138, where the lesion is likely to be visible. Alternatively, biopsy unit 116 and x-ray detector array 503 are positioned in any of a continuous range of positions relative to support base 138. However, it is difficult to be certain where the x-ray detector should be positioned in order to see the lesion, since the breast is typically more highly compressed when making a mammogram than when doing a biopsy, distorting its shape, and the breast may be compressed from a different angle when doing the biopsy, depending on the planned angle of approach of the biopsy needle. If the first digital x-ray image, made of the breast with the x-ray detector at a chosen position, does not show the lesion, then the x-ray detector and biopsy unit are moved to a different position, corresponding to an adjacent zone, for example, and another digital x-ray image is made. This procedure is repeated until the lesion is seen in the digital x-ray image.

Optionally, a flat compression plate 618 is used in addition to a soft compression device, as shown in FIGS. 6B and 6C. FIG. 6B is a side view, like FIG. 5A, but including compressed breast 602, and including flat compression plate 618. FIG. 6C is perspective view of the biopsy unit without the support base or the soft compression device, like FIG. 5B, and including flat compression plate 618. The breast is first compressed in soft compression device 128, which is attached to support base 138. The X and Y coordinates of biopsy unit 116 are then adjusted, using the XY positioner, as described previously, so that detector array 503 is below the lesion. The position of the lesion is optionally verified by a preliminary x-ray image. Biopsy needle 504, directly above detector array 503, is within the range of its motion above the lesion.

Then flat compression plate 618, made of polycarbonate for example, and attached to the biopsy unit, is used to further compress the breast, as shown in FIG. 6B. Stereotactic x-rays are then made, as described previously, and the biopsy is performed through a window 620 in flat compression plate 618. The window is optionally the same size as detector array 503, and directly over it. The flat compression plate optionally extends far enough in the X and Y directions so that it can compress and hold the breast, with the window situated over any of the zones shown in FIG. 6A for example, and not only if window 620 is over a center zone of the breast.

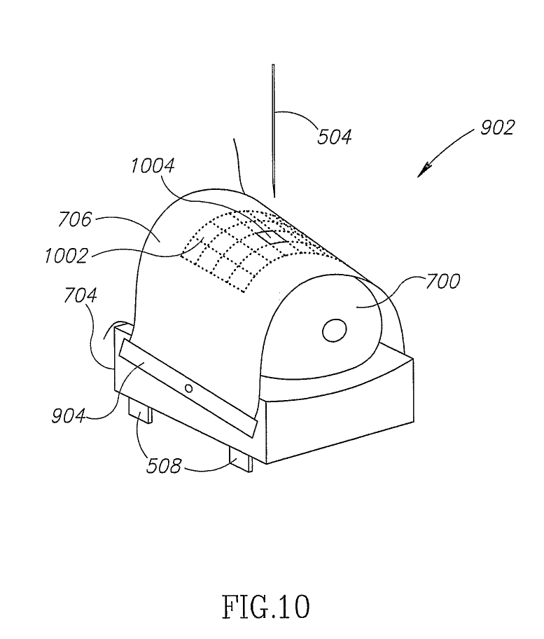

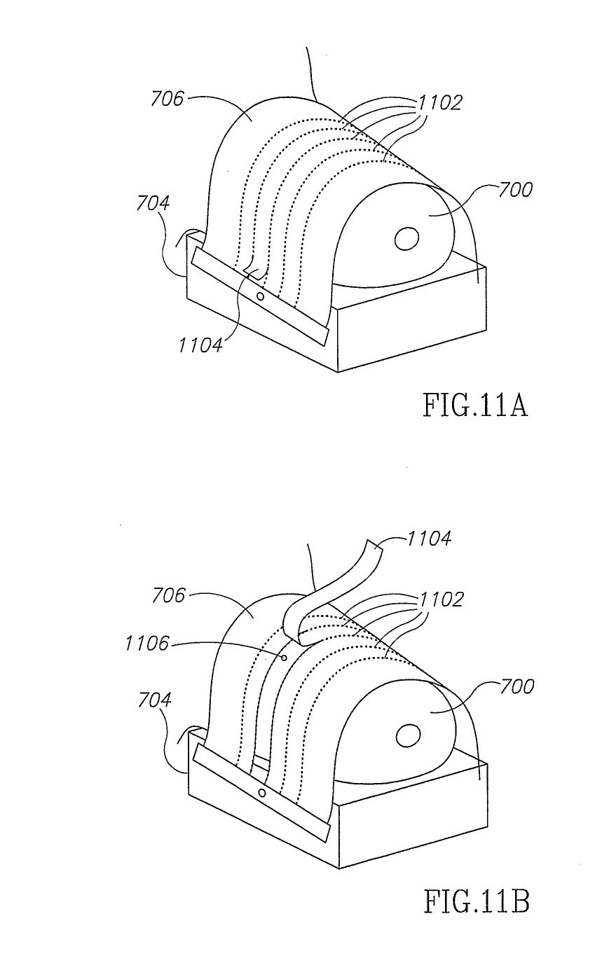

Optionally, the biopsy needle penetrates soft compression device 128 when the biopsy is done through window 620. Alternatively, a portion of the soft compression device is opened up and optionally removed, before doing the biopsy, for example a rectangular area or a long strip, as described below in the description of FIGS. 10, 11A and 11B. When the soft compression device is used together with a hard compression device as shown in FIGS. 6B and 6C, then preferably the opening is made in the soft compression before the breast is further compressed in the hard compression device, since it may be difficult to open up or remove a portion of the soft compression device after the breast is further compressed in the hard compression device. In this case, the exposed skin of the breast is optionally sterilized after the hard compression device is applied, through window 620.

Using flat compression plate 618 together with soft compression device 128 is potentially especially advantageous in the case of a patient with small breasts, which may be difficult to immobilize completely enough, or to compress strongly enough, to perform a biopsy, using the soft compression device alone. Instead, the soft compression device compresses the breast sufficiently so that the patient can lean back, and the zone where the lesion is located can be verified. Without any need for the patient to sit upright again, flat compression plate 618 then compresses the breast further, so that good quality stereotaxic x-rays can be made, and so the breast is immobile enough to perform a biopsy using the stereotaxic x-rays.

Optionally, if the compression device is one that allows ultrasound imaging of the breast to be used, then ultrasound imaging is used to guide the biopsy, in addition to or even instead of x-ray images, before the biopsy begins and/or during the biopsy. Optionally, x-ray images are used to guide the initial approach of the biopsy needle to the lesion, and ultrasound images are used to guide the final approach.

As in conventional biopsy systems, the biopsy needle has some degrees of freedom in its position, relative to the biopsy unit. The fact that the whole biopsy unit can rotate by a relatively large angle relative to the breast, optionally as much as 270 degrees or even more, and can move further from and closer to the breast (the X direction of the XY positioner), increases the range of angles and positions from which the biopsy needle can enter the breast. This potentially allows the biopsy needle to reach regions, for example close to the chest wall, which might be difficult or impossible to reach with conventional biopsy systems, and it may allow the biopsy needle to travel a shorter distance through breast tissue, producing less trauma and better healing of the biopsy wound. If the patient is in a reclined or a completely supine position, then the other breast may tend to fall back, making it easier for the biopsy needle to approach the breast from an angle between the two breasts, if the lesion is located on that side of the breast.

Optionally, the chair and x-ray unit shown in FIGS. 1-4A are used for making x-ray images of the breast, for example diagnostic x-ray images, without performing a biopsy. Optionally, there is no biopsy unit, but there is an x-ray detector which the compression device is adapted to couple to in a stable way, so that x-ray images of the breast may be made. As in the case of x-ray images made for guiding a biopsy, the compression device may be one of the soft compression devices described below in FIGS. 7, 8 and 9, or a conventional breast compression device. If the x-ray images are to be used for diagnostic purposes, then optionally the compression device compresses the breast with a relatively high force, for example 12 kilograms or greater. Such high forces are generally not needed for x-ray guided biopsies.

FIG. 7 is a front view of a breast 700 in a soft compression device 702. The breast is inserted into the compression device preferably when the patient is sitting upright. A relatively rigid base 704 goes under the breast, and a flexible band 706, made of nylon for example, goes at least part way around the breast, optionally at least half way around the breast, and is attached at both ends to base 704. A tensioning device 708, located at at least one end of flexible band 706, is then tightened, to hold the breast firmly in the compression device, so that it does not slip out or move significantly as the patient is placed in a reclining or supine position, but preferably not so tight as to be uncomfortable. For this reason, soft compression device 702 is particularly suitable to be used with one of the reclining chair biopsy systems shown in FIGS. 1-6C, but it may also be used with other breast biopsy systems. Optionally, flexible band 706 is elastomeric, allowing it to conform its shape to some extent to the surface of the breast, and hence distributing the compressive force more uniformly over the surface of the breast in the axial direction.

Coupling mechanism 508 is used to lock the base of the compression device to the support base when the compression device is used with one of the biopsy systems shown in FIGS. 1-6C, as described above. As explained previously, although coupling mechanism 508 is shown in the drawings as tabs that fit into slots, this is merely illustrative, and any rigid lockable coupling mechanism known in the art may be used.

Optionally, when tensioning device 708 is tightened, the compression device exerts a compressive force of less than 1 kilogram on the breast, or between 1 and 2 kilograms, or between 2 and 5 kilograms, or between 5 and 10 kilograms, or more than 10 kilograms. Although these forces may not be sufficient for making diagnostic quality x-ray images of the breast, they may be adequate for making x-ray images for guiding a biopsy. Optionally, the compression device exerts a compressive force on the breast that is sufficient for diagnostic x-ray images of the breast, for example between 10 and 12 kilograms, or between 12 and 15 kilograms, or between 15 and 20 kilograms, or more than 20 kilograms. These ranges of force also optionally apply to the compression device shown in FIG. 9, as well as to conventional breast compression devices optionally used with the chairs shown in FIGS. 1-4C.

Optionally, the tension device works by shortening the flexible band, for example by rolling it up, or by buckling it like a belt, or by acting like the buckles with teeth used to tighten the straps on a backpack, or by pulling it through a gap between a roller and another surface that the roller is pressed against, and not allowing the roller to turn back unless released, or by any other means known to the art.

Optionally, the base has a flat top. Alternatively, the base is curved on top, to fit the breast, as shown in FIG. 7. In this case, the base optionally comes in a plurality of different sizes, for use with breasts of different sizes.

Optionally, an x-ray detector is incorporated into base 704, instead of being permanently attached to the biopsy unit. However, if the x-ray detector is a digital array which covers only 50 mm by 50 mm, then there is optionally a way to change its position relative to the base, in order to detect a lesion anywhere in the breast. Also, if the x-ray detector is used to guide a biopsy, as opposed to just being used for diagnostic x-rays for example, then optionally there is a way to communicate to the biopsy unit where the x-ray detector is located in the base. For example, there are optionally one or more reference marks, at fixed locations with respect to the biopsy unit, and located at positions such that at least one reference mark is visible in the x-ray image for any location of the x-ray detector. Also, if the x-ray detector is used to guide a biopsy, then the robot arm moving the biopsy needle optionally has a range of motion in X and Y great enough to cover the whole breast, instead of just a 50 mm by 50 mm range of motion.

The base, including the coupling mechanism, is preferably rigid enough so that the breast remains at the same position relative to the support base, when the compression element is locked to the support base, from the time that images are made of the breast to guide the biopsy, until the biopsy is completed.

Although the base is shown under the breast in FIG. 7, optionally it goes above the breast, or on either side of the breast, at any angle. In particular, as explained previously, it is optionally oriented at different angles, depending on the desired angle of approach of the biopsy needle.

Optionally, ultrasound imaging is used to image the breast, before and/or during the biopsy, to guide the biopsy, if the lesion is of a type that is visible with ultrasound. The flexible band is thin enough and has a density and elastic modulus such that ultrasound can penetrate the band, and can couple well from the band into the breast as long as there is no air gap between the elastic band, the breast and the ultrasound detector. FIG. 7 shows an ultrasound transducer 710, hand-held for example, pressed indirectly against the breast through flexible band 706, which transducer is used to transmit and receive ultrasound waves, to produce ultrasound images of the breast. Because the flexible band goes a significant amount around the breast, for example half way around or more, there is a wide range of the breast surface against which an ultrasound transducer can be placed, indirectly through the flexible band. Alternatively, if a portion of the flexible band, for example a rectangle or long strip, is opened up or removed before performing the biopsy, as described below in the description of FIGS. 10, 11A, and 11B, then ultrasound imaging is done directly through the exposed skin.

FIG. 8 is a side view of the breast in soft compression device 702, and FIG. 9 is a side view of a breast in a different soft compression device 902. In soft compression device 702, as seen in FIG. 8, flexible band 706 is always oriented with its width parallel to the axis 802 of the breast. Hence, depending on the shape of the breast, soft compression device 702 will generally compress the breast with greater force near the chest wall, and with less force near the nipple, and a large part of the area of the flexible band may not even make contact with the surface of the breast at all. This may make the soft compression device uncomfortably tight, and/or may make it less effective at stabilizing and compressing the breast.

Compression device 902 in FIG. 9 at least partially overcomes these problems, by allowing the angle at which flexible band 706 joins base 704 to vary, depending on the shape of the breast. An anchoring element 904, where flexible band 706 joins base 702, may be rotated, so that its angle matches the angle of the breast. The rotation is around what an aeronautical engineer would call the “pitch axis,” extending to the left and right of the breast, as opposed to the roll or yaw axis. The pitch axis is normal to the plane of the drawing in FIG. 9. This rotation ensures that flexible band 706 is largely in contact with the breast, unlike the case show in FIG. 8. Optionally, anchoring element 904 also serves as tensioning device 708, described in FIG. 7.

Optionally, anchoring element 904 is fitted with a spring, which makes it automatically find an optimal or near optimal angle of orientation, so that flexible band 706 exerts a relatively uniform pressure on the breast, over a relatively large fraction of the surface of the flexible band. Alternatively or additionally, the angle may be adjusted manually, until it appears to be optimal, and anchor 904 is then optionally locked into place.

Optionally, tensioning device 708, whether or not it is identical to anchoring element 904, allows flexible band 706 to be adjusted so that it forms part of the surface of a cone. This ability provides an additional degree of freedom for conforming flexible band 706 to the surface of the breast, and producing a more uniform pressure. When tensioning flexible band 706 around the breast, it may also sometimes be desirable to make the surface less conical, and more like a tilted cylinder, in order to compress the breast more in the direction that the x-rays are coming from.

FIG. 10 shows a biopsy needle 504 approaching a breast 700, held by soft compression device 902, with flexible band 706 covering most of the breast. The features shown in FIG. 10 could also be used with compression device 702. Flexible band 706 has a grid 1002 of perforations, for example a rectangular grid, or a square grid with the individual squares 1 cm on a side, or 5 mm on a side, or anything in between, or larger than 1 cm or smaller than 5 mm.

Once it has been determined where the needle will enter the breast, a square 1004 at the proper location is torn off, using the perforations. The exposed skin that was beneath that square is then sterilized, and the needle enters the breast under sterile conditions. Alternatively, for example if there are no perforations, the needle penetrates through the flexible band into the breast. In that case, the entire surface of the breast is preferably sterilized before the breast is inserted into the soft compression device, and the soft compression device itself, or at least the flexible band, is sterilized, and care is taken to keep it sterile when it is fitted around the breast. A potential advantage of using the perforations is that is not necessary to sterilize the entire surface of the breast, or the flexible band, but only the small area that is exposed before the biopsy needle penetrates the skin.

Alternatively, as shown in FIG. 11A, instead of a grid of perforations there are a plurality of rows of perforations 1102, parallel or at least roughly parallel to each other, extending over the length of flexible band 706. Alternatively, some or all of the rows do not extend over the whole length of flexible band 706, but extend to one end of flexible band 706, where it joins base 704. To expose a portion of the breast for performing the biopsy, a sharp instrument such as a knife is used to begin cutting off a strip 1104 of the flexible band at the end of the flexible band, where it joins the base. The strip is bounded by two of the rows of perforations which extend to that end of the flexible band. The sharp instrument will not accidentally cut the breast, since the flexible band is not in contact with the breast in the region where the flexible band joins the base, as may be seen in FIG. 7. Once strip 1104 has been cut off for a short distance, it can continue to be torn off by hand, because of the perforations, as shown in FIG. 11B. Strip 1104 is optionally torn off up to a short distance past a point 1106 where the biopsy needle is to enter the breast. Strip 1104 is then optionally cut off flexible band 706, to avoid having it get in the way, or alternatively is simply turned back or folded back, as shown in FIG. 11B. The exposed skin is then sterilized, at least in the vicinity of point 1106, and the biopsy is performed.

A potential advantage of the using the device and procedure shown in FIGS. 11A and 11B is that it may be difficult to tear off a square of the flexible band by hand, even with a grid of perforations, if the square is in the middle of the flexible band and the flexible band is tightly pressed against the breast. The procedure shown in FIGS. 11A and 11B avoids this potential difficulty, while retaining many of the potential advantages of the device and procedure described for FIG. 10.

The invention has been described in the context of the best mode for carrying it out. It should be understood that not all features shown in the drawings or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.” As used herein, “coupled” can mean directly or indirectly coupled mechanically, as well as constrained to move together, or controlled to move together, for example by software.

1. A breast compression device suitable for compressing a breast for imaging, the device comprising:
a) a base for contacting a portion of the surface of the breast;
b) a flexible band, anchored to the base, adapted to be positioned on a side of the breast opposite the base, when the breast is inserted between the base and the band, which band is adapted to be wrapped at least part way around the breast; and
c) a tensioning device, adapted to tension the flexible band around the inserted breast when the flexible band is anchored to the base, thereby compressing the breast between the flexible band and the base.

2. A device according to claim 1, wherein the flexible band is adapted to be wrapped at least half way around the breast.

3. A device according to claim 1, wherein the base is relatively rigid.

4. A device according to claim 1, wherein the base is flat where it contacts the breast.

5. A device according to claim 1, wherein the base is curved to conform to the breast.

6. A device according to claim 1, adapted to compress the breast with a force between 1 and 12 kilograms.

7-8. (canceled)

9. A device according to claim 1, which does not cover a region around the nipple when compressing the breast.

10. A device according to claim 1, wherein the flexible band comprises nylon.

11. A device according to claim 1, wherein the flexible band is elastomeric.

12. A device according to claim 1, wherein the flexible band is capable of being punctured by a biopsy needle.

13. A device according to claim 1, wherein the flexible band is adapted to having an area of the band removed, thereby exposing skin in that area and enabling a biopsy to be performed by puncturing the skin in that area.

14. A device according to claim 1, configured so that the flexible band wraps a distance around the breast that is different for different positions along the axis of the breast, thereby allowing the compression device to better fit the contour of the breast and to compress it more uniformly.

15. A device according to claim 14, and including an anchor which attaches one or both ends of the flexible band to the base, which anchor is capable of rotating, relative to the base, around an axis extending to the left and right of the breast.

16. A system for performing a breast biopsy, the system comprising:
a) a breast compression device according to claim 1;
b) an imaging unit adapted to obtain images of the breast when the breast is compressed by the compression device; and
c) a biopsy element capable of being directed to a lesion in the breast, guided at least in part by the images.

17. A system according to claim 16, wherein the imaging unit is an ultrasound imaging unit adapted to obtain the ultrasound images by one or both of transmitting and receiving ultrasound waves through the compression device.

18. A system for x-raying the breast, the system comprising:
a) a breast compression device according to claim 1; and
b) an x-ray unit and an x-ray detector adapted for producing x-ray images of the breast when the breast is compressed by the compression device.

19. A system according to claim 18, also including a biopsy unit adapted to be coupled to the compression device in a spatially stable way, with a biopsy element capable of being directed to a lesion in the breast, guided by the x-ray images.

20. A system according to claim 19, also including a controller, wherein the x-ray detector and x-ray images are digital, the x-ray detector is adapted to send the x-ray images to the controller, and the controller is adapted to use the x-ray images to calculate the three-dimensional location of the lesion and to direct the biopsy element to said location.

21. A system according to claim 19, also including an ultrasound imaging unit adapted to obtain ultrasound images of the breast when the breast is compressed by the compression device, wherein the biopsy element is capable of being guided in its initial approach to the lesion by the x-ray images and in its final approach to the lesion by the ultrasound images.

22. A system according to claim 18, also including a chair with a back that has an upright position and at least one reclining position, wherein the compression device is adapted to hold the breast of a patient seated in the chair in a compressed state and in a stable position and orientation relative to the x-ray detector, at least in the reclining position.

23-24. (canceled)

25. A system for performing a biopsy of a breast lesion of a patient in a reclining position, the system comprising:
a) a chair with a back that has an upright position and at least one reclining position;
b) a compression device adapted to hold a breast of the patient seated in the chair in a compressed state, when the chair back is in the upright position and when it is in at least one reclining position; and
c) a biopsy unit having a biopsy element and configured to remain in substantially the same position and orientation relative to the breast of the patient when the chair back goes from the upright position to the at least one reclining positions, thereby enabling the biopsy to be performed in any of the at least one reclining positions.

26. A system according to claim 25, and also including an x-ray unit, configured to remain in substantially the same position and orientation relative to the breast of the patient when the chair back goes from the upright position to the at least one reclining positions, thereby enabling x-ray images to be made to guide the biopsy, in any of the at least one reclining positions.

27. A system according to claim 26, wherein the chair is mounted on a base adapted to rest on a floor, and the x-ray unit and the biopsy unit are mounted in a stable position relative to the base of the chair.

28. A system according to claim 25, wherein the biopsy unit is adapted to be coupled to the compression device in a manner allowing the biopsy unit to remain in a fixed position relative to the compression device.

29. A system according to claim 28, wherein the biopsy unit is adapted to be rigidly coupled to the compression device.

30. A system according to claim 28, wherein the relative position of the biopsy unit and the compression device is sufficiently stable to allow the biopsy element to be directed to the lesion when the breast is held in the compression device in the compressed state, guided by x-ray images of the breast made when the breast is held in the compression unit in the compressed state.

31. A system according to claim 30, wherein the compression device comprises:
a) a soft compression device according to claim 1, adapted to hold a breast of the patient seated in the chair in a compressed state, when the chair back is in the upright position or in at least one reclining position; and
b) a hard compression device, adapted to further compress the breast when the breast is already held in the soft compression device and the chair back is in the reclining position, thereby enabling said x-ray images, if made when the breast is compressed by the hard compression device, to be of sufficient quality to guide the biopsy.

32. A system according to claim 31, wherein the hard compression device comprises a flat, relatively rigid plate and wherein the biopsy unit is adapted to perform the biopsy through a window in the hard compression device.

33. (canceled)

34. A system according to claim 25, and including an x-ray detector coupled to one or both of the biopsy unit and the compression device, in a sufficiently stable way so as to enable the x-ray detector to be used for making x-ray images of the breast to guide the biopsy.

35. (canceled)

36. A system according to claim 34, wherein the x-ray detector has a field of view that does not include the entire breast, and the x-ray detector is adapted to move to a plurality of positions, thereby enabling any of a plurality of zones of the breast to fall within its field of view, while the x-ray detector remains related to one or both of the biopsy unit and the compression device in the sufficiently stable way.

37. A system according to claim 36, wherein the x-ray detector and the biopsy unit move together maintaining a same relative position, when the x-ray detector moves to a different one of the plurality of positions.

38. A system according to claim 25, and including an x-ray unit coupled to the chair at least in a reclining position.

39. A system according to claim 25, wherein the biopsy unit is capable of being rotated into any of at least two different orientations, thereby enabling the biopsy element to approach the lesion from any of at least two different directions, depending on the location of the lesion.

40. A system for making x-ray images of a patient's breast, the system comprising:
a) a chair with a back that has an upright position and at least one reclining position;
b) a compression device adapted to hold a breast of a patient seated in the chair in a compressed state, when the chair back is in the upright position and when it is in at least one reclining position; and
c) an x-ray unit capable of being coupled to the chair back so that it is aimed at the breast when the patient is seated in the chair, at least when the chair back is in a reclining position.

41. A system according to claim 40, and including an x-ray detector coupled to the compression device, in a sufficiently stable way so as to enable the x-ray detector to be used for making x-ray images of the breast.

42. A system according to claim 41, wherein the x-ray detector is capable of being oriented in any of at least two different angles relative to the breast, thereby enabling the x-ray images to be made from any of at least two different angles depending on the location of the lesion.

43. A system according to claim 40, wherein the x-ray unit is coupled to the back of the chair, such that the x-ray unit remains in substantially the same position and orientation relative to the breast of the patient when the chair goes from the upright position to the at least one reclining positions.

44. A system according to claim 40, wherein the x-ray unit is configured to move to either of two positions in which the x-ray unit directs x-rays at the breast of the patient from different directions, thereby enabling stereotaxic x-ray images of the breast to be made.

45. A system according to claim 42, wherein the angles from which the x-ray images are made at least 60 degrees apart.

46. A system according to claim 25, wherein the patient is leaning back by an angle of at least 10 degrees when the chair is in the reclining position.

47. A system according to claim 46, wherein the angle is at least 30 degrees.

48. A system according to claim 47, wherein the angle is at least 45 degrees.

49. (canceled)

50. A system according to claim 48, wherein the angle is at least 75 degrees.

51. A system according to claim 50, wherein the angle is approximately 90 degrees.

52. A method of imaging of a patient's breast, the method comprising:
a) seating the patient in a chair at least the back of which can lean back;
b) compressing the breast of the patient in a compression device;
c) causing the back of the chair to lean back such that the patient is reclining at an angle of at least 10 degrees; and
d) making x-ray images of the compressed breast when the patient is reclining at said angle.

53. A method according to claim 52, wherein compressing the breast in a compression device comprises compressing the breast in a soft compression device according to claim 1, before causing the back of the chair to lean back.

54. A method according to claim 53, wherein compressing the breast in a compression device also comprises compressing the breast further with a hard compression device, after compressing the breast in the soft compression device and causing the back of the chair to lean back, and before making the images, thereby enabling the x-ray images to be of sufficient quality to guide the biopsy.

55. A method according to claim 52, wherein the images are x-ray images and wherein making x-ray images comprises making a stereotaxic pair of x-ray images.

56. A method according to claim 55, also including:
a) coupling the compression device to a biopsy unit; and
b) performing a biopsy of a lesion in the breast, using a biopsy element in the biopsy unit, guided at least by the x-ray images.

57. A method according to claim 56, wherein coupling the compression device to the biopsy unit is done after compressing the breast.

58. A method according to claim 56, and including:
a) determining an at least approximate location of the lesion in the breast; and
b) choosing one or more of a direction at which the biopsy element enters the breast, an average direction from which the stereotaxic pair of x-ray images is made, and a direction in which the breast is compressed in the compression device, depending on said location of the lesion.

59. A method according to claim 58, wherein choosing one or more of the directions comprises choosing two or more of the directions to be approximately the same direction.

60. A method according to claim 56, and including:
a) determining whether the lesion is visible in at least one of the x-ray images;
b) if the lesion is not visible, moving the x-ray detector so that its field of view includes a different zone of the breast, and making a new x-ray image;
c) determining if the lesion is visible in the new x-ray image; and
d) repeating (b) and (c) until the lesion is visible in the new x-ray image.

61. A method according to claim 60, wherein moving the x-ray detector comprises moving the x-ray detector together with the biopsy unit, so that the x-ray detector and the biopsy unit maintain the same relative position.

62. (canceled)

63. A method of performing a biopsy on a lesion in the breast, the method comprising:
a) wrapping a flexible band at least part way around the breast;
b) anchoring the flexible band to a base;
c) compressing the breast between the flexible band and the base, by applying tension to the flexible band;
d) coupling one or both of the flexible band and the base to a biopsy unit, with sufficient spatial stability to enable the biopsy unit to perform an image guided biopsy;
e) making images of the breast while it is compressed between the flexible band and the base; and
f) performing the biopsy on the breast while it is so compressed, using the biopsy unit, guided at least by the images.

64. A method according to claim 63, in which wrapping a flexible band at least part way around the breast comprises wrapping the flexible band at least half way around the breast.

65. A method according to claim 63, wherein coupling one or both of the flexible band and the base to the biopsy unit comprises coupling the base to the biopsy unit.

66. A method according to claim 63, and including:
a) determining an at least approximate location of the lesion in the breast; and
b) positioning the base on a portion of the breast that depends on said location.

67. A method according to claim 63, wherein performing the biopsy comprises puncturing the flexible band with a biopsy element.

68. A method according to claim 67, including sterilizing a substantial part of the surface of the breast, and the flexible band, before wrapping the flexible band around the breast.

69. A method according to claim 63, and including removing an area of the flexible band from the breast before performing the biopsy, thereby exposing the skin in that area, wherein the biopsy is performed by puncturing said exposed skin by a biopsy element.

70. (canceled)

71. A method according to claim 63, wherein performing the biopsy is done while the patient is reclining back by an angle of at least 10 degrees from the vertical.

72. A method according to claim 52, wherein said angle is at least 30 degrees.

73. A method according to claim 72, wherein said angle is at least 45 degrees.

74. (canceled)

75. A method according to claim 73, wherein said angle is at least 75 degrees.

76. A method according to claim 75, wherein the angle is approximately 90 degrees.

77. A method according claim 52, wherein compressing the breast is done with a force between 1 and 12 kilograms.

78-79. (canceled)

80. A system according to claim 25 wherein the biopsy unit is coupled to the chair back, in at least one of said reclining positions.

81. A system according to claim 28 wherein the biopsy unit is not rigidly coupled to the compression device.
